(19) United States
(12) Patent Application Publication
(10) Pub. No.: US 2018/0274037 A1
(43) Pub. Date: Sep. 27, 2018

(54) NOVEL BIOMARKERS FOR PANCREATIC DISEASES

(71) Applicant: Queen Mary University of London, London (GB)

(72) Inventors: Tatjana CRNOGORAC-JURCEVIC, London (GB); Silvana BERNARDI, London (GB)

(73) Assignee: Queen Mary University of London, London (GB)

(21) Appl. No.: 15/762,275

(22) PCT Filed: Sep. 26, 2016
(86) PCT No.: PCT/GB2016/052991

§ 371 Date: Mar. 22, 2018

(30) Foreign Application Priority Data
Sep. 25, 2015 (GB) 1517028.5

(51) Int. Cl. C12Q 1/6886

(52) U.S. Cl. CPC C12Q 1/6886; C12Q 2600/158; C12Q 2600/178

(57) ABSTRACT

The present invention relates to miRNA biomarkers for use in the diagnosis of pancreatic diseases, in particular pancreatic cancers and pre-cancerous diseases, such as pancreatic ductal adenocarcinoma (PDAC), in particular the early detection of PDAC. The miRNA biomarkers are miR-143, miR-223, miR-204, miR-30e, miR-26a, miR-30a, miR-30b and miR-106b. They may be combined with protein biomarkers. The present invention also provides methods of diagnosis and treatment of PDAC and related diseases, and kits for the early detection of PDAC based on the expression levels of the biomarkers in biological samples, in particular urine samples.

The present invention relates to pancreatic diseases, in particular pancreatic cancers and pre-cancerous diseases, and the use of biomarkers in biological samples for the diagnosis of such conditions, in particular early stage pancreatic ductal adenocarcinoma (PDAC). The present invention also relates to the use of biomarkers in biological samples for the diagnosis of CP, and the classification of pancreatic disease.

PDAC is the most common exocrine pancreatic malignancy accounting for more than 80% of malignant neoplasms arising in pancreas. It is the fourth most common cause of cancer-related deaths in the Western world. When diagnosed, the majority of patients display locally advanced disease or have established metastases, therefore surgery is possible in only 10-20% of patients (Sener et al. (1999) J Am Coll Surg, 189:1-7). While the overall 5-year survival rate is less than 5%, the 5-year survival in patients after surgical resection and adjuvant chemotherapy can reach 30%.

Pancreatic ductal adenocarcinoma (PDAC) is one of the rare cancers for which no significant improvements in the diagnostic and therapeutic approaches have been made in the last decades. Despite considerable progress in our understanding of the disease at the molecular level, novel findings have not yet reached the clinic, and the majority of patients are still faced with a grim average survival of 5 to 6 months. With over 38,000 PDAC-related deaths in the US and over 40,000 in Europe in 2013, this malignancy is currently the fourth leading cause of cancer-related death, but predicted to become the second by 2030.

PDAC is one of the most challenging cancers to detect. The retroperitoneal position of the pancreas, a number of complex underlying molecular abnormalities, and the lack of specific clinical symptoms result in a majority of patients presenting at an advanced stage. Fewer than 20% of patients can thus undergo potentially curative surgery, while the remaining ones can only be offered palliative treatment.

These worrying figures would change significantly with improved tools for early detection, as 5-year survival approaching 70% has been reported after incidental diagnosis of stage I PDAC tumours, when they were still confined to the pancreas with a size <2 cm. Importantly, a considerable ‘window’ of opportunity of around a decade exists for earlier diagnosis (Yachida S, et al. (2010) Nature 467(7319):1114-1117). Detection at an early stage is also crucial given the poor efficacy of current therapies for metastatic disease, when potentially curative surgery is no longer feasible.

Timely detection of PDAC is, however, hampered by several factors: lack of specific clinical symptoms in the early stage of the disease, insufficient sensitivity of current imaging modalities and, despite intensive efforts, lack of accurate body fluid-based biomarkers of early-stage disease (for a review see Kaur et al. (2012) Biomark Med 6(5):597-612.). Early stage PDAC is also difficult to differentiate from chronic pancreatitis (CP), a benign inflammatory disease of the pancreas and one of the risk factors for PDAC. Serum carbohydrate antigen 19.9 (CA19.9), the only PDAC biomarker in widespread clinical use at present, suffers from false negative results in patients with Lewis-negative genotype, poor positive predictive value in the asymptomatic population and low sensitivity (79%-81%) in symptomatic patients. Less than 50% of cases with early disease (tumour <2 cm) have raised CA19.9 levels, yet CA19.9 levels may be elevated in various other benign and malignant pancreatic and hepato-biliary diseases (including chronic pancreatitis), as well as in unrelated cystic and inflammatory diseases (for review see Ballehaninna & Chamberlain (2011) Indian J Surg Oncol, 2:88-100). In addition, Lewis a/b antigen (which Ca19.9 recognizes) is not expressed in around 10% of population.

Proteomic techniques have recently been used to study protein expression in pancreatic cancer tissue, pancreatic juice and serum/plasma specimens (see, for example, Koomen et al. (2005) Clin Cancer Res, 11:1110-1118), but none of these have, as yet, resulted in the discovery of biomarkers suitable for clinical practice.

Recently, urine was studied as a potential source of biomarkers as it is an easily and non-invasively obtained bio-fluid (Pieper et al. (2004) Proteomics, 4:1159-1174). In comparison with plasma, urine proteins are less complex and more thermostable. Furthermore, most common proteins (albumin, uromodulin) comprise a lesser proportion of the urinary proteome, so sample processing requires less pre-cleaning/fractionation. Approximately 49% of urinary proteins are soluble products of glomerular filtration of plasma (Barrat et al. (2007) Cmaj, 177:361-368), and therefore a substantial number of proteins in urine arise from extrarenal sources (Thongboonkerd et al. (2005) Curr Opin Nephrol Hypertens, 14:133-139).

MicroRNAs (miRNAs), small non-coding evolutionarily conserved RNAs, are critically implicated in the regulation of a whole host of cellular processes, and their aberrant expression is associated with cancer in a variety of tissues, including pancreas (Roldo C et al. (2006) J Clin Oncol, 24:4677-4684 and Bloomston M et al. (2007) JAMA, 297:1901-1908). Their quantification in body fluids has a huge diagnostic potential as it has already been demonstrated for several solid tumors, including PDAC. However, the high specificity and sensitivity of circulating miRNAs for the early detection of PDAC has not been achieved, even when combined with serum CA19.9 (Liu J et al. (2012) Int J Cancer, 131:683-691, and Schultz N A et al. (2014) JAMA, 311:392-404).

Munding et al., Int J Cancer, discloses global microRNA expression profiling of microdissected pancreatic tissues. Mall et al. (2013) Biomark Med, 7(4):623-631 discusses stability of miRNAs in urine.

In addition to urological cancers, several cancer-related proteins have been identified in the urine of patients with lung, ovarian and breast cancers. WO2004/102189 describes biomarkers for the diagnosis pancreatic cancer. Serum samples from patients with pancreatic cancer were compared with serum samples from healthy donors and the resulting biomarkers characterized by their weight. A similar approach was carried out in WO2004/099432, which provides further biomarkers for detecting pancreatic cancer.

Due to the late diagnosis and the aggressive nature of pancreatic adenocarcinoma (PDAC), median survival of patients with the disease is usually 5-6 months and five-year survival <5%. Highly accurate biomarkers for early detection are thus expected to significantly impact patients' prognosis.

There remains a need for a sensitive and specific panel of markers that would enable not only early diagnosis of PDAC, but also aid in differentiating between PDAC and other tumours, as well as between PDAC and chronic pancreatitis (CP) and pancreatic cystic lesions. Preferably the markers will be detectable in a sample that is easy and non-invasive to obtain and is sensitive enough to detect the disease during its early stages.

### SUMMARY OF THE INVENTION

In a first aspect of the invention there is provided a biomarker panel useful in the diagnosis of pancreatic ductal adenocarcinoma (PDAC), in particular early stage PDAC, the panel comprising miR-143, miR-223, miR-204, miR-30e, miR-26a, miR-30a, miR-30b and/or miR-106b. In some embodiments, the panel may further comprise the biomarker proteins LYVE1, REG1 (for example REG1A and/or REG1B) and/or TFF1, and/or the panel may further comprise the biomarker protein CA19.9.

In a second aspect of the invention there is provided a method of diagnosing, screening or testing for PDAC, in particular early stage PDAC, comprising detecting the level of expression or concentration of an miRNA selected from the group consisting of miR-143, miR-223, miR-204, miR-30e, miR-26a, miR-30a, miR-30b and miR-106b, or combinations thereof, in a biological sample. In some embodiments, the biological sample is a urine sample. Methods of treatment of PDAC, in particular early stage PDAC, in patients using these biomarkers as diagnostic indicators are also provided.

In a third aspect of the invention there is provided an miRNA selected from the group consisting of miR-143, miR-223, miR-204, miR-30e, miR-26a, miR-30a, miR-30b and miR-106b, or a combination thereof, for use in diagnosing PDAC, in particular early stage PDAC. There is also provided the use of a miRNA selected from the group consisting of miR-143, miR-223, miR-204, miR-30e, miR-26a, miR-30a, miR-30b and miR-106b, or a combination thereof, in methods of detecting or diagnosing early stage PDAC.

In a fourth aspect of the invention there is provided an miRNA selected from the group consisting of miR-143, miR-223, miR-204, miR-30e, miR-26a, miR-30a, miR-30b and miR-106b, or a combination thereof, for use in diagnosing chronic pancreatitis (CP). There is also provided the use of a miRNA selected from the group consisting of miR-143, miR-223, miR-204, miR-30e, miR-26a, miR-30a, miR-30b and miR-106b, or a combination thereof, in methods of detecting or diagnosing CP. Methods of treatment of CP in patients using these biomarkers as diagnostic indicators are also provided. miR-26a, miR-30a, miR-30b and miR-106b may be particularly use in methods relating to CP.

In a fifth aspect of the invention there is provided a kit for testing for pancreatic ductal adenocarcinoma, in particular early stage PDAC, comprising a means for detecting the level of expression or concentration of an miRNA selected from the group consisting of miR-143, miR-223, miR-204, miR-30e, miR-26a, miR-30a, miR-30b and miR-106b, or combinations thereof, in a biological sample. The kit may be a biosensor (or any other means of detection), and may comprise instructions for use.

In a sixth aspect of the invention there is provided a kit for testing for chronic pancreatitis, comprising a means for detecting the level of expression or concentration of an miRNA selected from the group consisting of miR-143, miR-223, miR-204, miR-30e, miR-26a, miR-30a, miR-30b and miR-106b, or combinations thereof, in a biological sample. The kit may be a biosensor (or any other means of detection), and may comprise instructions for use. miR-26a, miR-30a, miR-30b and miR-106b may be particularly use in kits relating to CP.

In a further aspect of the invention there is provided a method of distinguishing between PDAC, in particular early stage PDAC, and chronic pancreatitis, comprising detecting the level of expression or concentration of miR-143, miR-223, miR-204, miR-30e, miR-26a, miR-30a, miR-30b and/or miR-106b in a urine sample and comparing the expression levels of each of the quantified miRNAs with a reference. In these embodiments, the use of miR-223 and/or miR-204 may be preferred. In some embodiments, the use of miR-30b and/or miR106b may be preferred. Kits for conducting such a differential diagnosis are also provided.

In a further aspect of the invention there is provided a method of distinguishing between CP and healthy patients in which no CP is present, comprising detecting the level of expression or concentration of miR-143, miR-223, miR-204, miR-30e, miR-26a, miR-30a, miR-30b and/or miR-106b in a urine sample and comparing the expression levels of each of the quantified miRNAs with a reference. In these embodiments, the use of miR-30b, miR-106b, miR-26a and/or miR-30b may be preferred. Kits for conducting such a differential diagnosis are also provided.

In a further aspect of the invention there is provided a method of diagnosing or detecting early stage PDAC, for example stage I PDAC, including distinguishing between stage I PDAC and healthy patients. The method comprises detecting the level of expression or concentration of the biomarker panel miRNAs miR-143, miR-223 and miR-30e in a biological sample and comparing the expression levels or concentration of each of the quantified miRNAs with a reference. In these embodiments, the use of miR-143 may be preferred, or the combination of miR-143 and miR-30e.

In a still further aspect of the invention there is provided a method of diagnosing or detecting early stage PDAC, for example stage I PDAC, including distinguishing between stage I PDAC and chronic pancreatitis. The method comprises detecting the level of expression or concentration of the biomarker panel miRNAs miR-143, miR-223 and miR-30e in a biological sample and comparing the expression levels or concentration of each of the quantified miRNAs with a reference. In these embodiments, the use of miR-223 and miR-204 may be preferred. In other embodiments, the use of miR-30b and/or miR106b may be preferred.

In a still further aspect of the invention there is provided a method of treating PDAC, in particular early stage PDAC, in a patient, comprising detecting the level of expression or concentration of an miRNA selected from the group consisting of miR-143, miR-223, miR-204, miR-30e, miR-26a, miR-30a, miR-30b and miR-106b, or combinations thereof, in a biological sample, comparing the level of expression with a control, and proceeding with treatment for PDAC if PDAC is diagnosed or suspected.

In a further aspect of the invention, there is provided a method of treating PDAC, in particular early stage PDAC, in a patient, comprising administering a treatment for PDAC to the patient, wherein the patient has been diagnosed as having PDAC according to a method of the invention.

In a still further aspect of the invention there is provided a method of treating CP in a patient, comprising detecting the level of expression or concentration of an miRNA selected from the group consisting of miR-143, miR-223, miR-204, miR-30e, miR-26a, miR-30a, miR-30b and miR-106b, or combinations thereof, in a biological sample, comparing the level of expression with a control, and proceeding with treatment for CP if CP is diagnosed or suspected. miR-26a, miR-30a, miR-30b and/or miR-106b may be particularly use in methods relating to the treatment of CP.

In a further aspect of the invention, there is provided a method of treating CP in a patient, comprising administering a treatment for CP to the patient, wherein the patient has been diagnosed as having CP according to a method of the invention.

Methods of prognosis are also included in the present invention, comprising determining the level of expression or concentration of one or more miRNAs selected from the group consisting of miR-143, miR-223, miR-204, miR-30e, miR-26a, miR-30a, miR-30b and miR-106b in a biological sample, comparing the level of expression with a control, and determining the prognosis for the patient.

In embodiments of the invention, the biomarkers used in the invention can be used separately (i.e. only one of miR-143, miR-223, miR-204, miR-30e, miR-26a, miR-30a, miR-30b or miR-106b), they can be used in pairs (for example miR-143 and miR-30e, or miR-223 and miR-204, or miR-26a and miR-30a, or miR-30b and miR-106b), or three may be used (for example miR-143, miR-223 and miR-30e, or miR-143, miR-223 and miR-204), or even four may be used (for example miR-26a, miR-30a, miR-30b and miR-106b). The specific context may determine which biomarkers or combination of biomarkers are used.

The present invention represents the first utility of miRNA biomarkers for non-invasive, early detection of PDAC in urine specimens. In any aspects of the invention, the miRNAs may be combined with other suitable biomarkers for pancreatic cancer. In particular, the miRNAs may be combined with analysis of protein biomarkers in a biological sample (such as a urine sample). In some embodiment, the protein biomarkers are selected from the group consisting of LYVE1, REG1 (REG1A and/or REG1B) and TFF1, and combinations thereof. The protein biomarker panel may further comprise CA19.9.

### BRIEF DESCRIPTION OF THE FIGURES

Reference is made to a number of Figures as follows:

FIG. 1: Hierarchical cluster analysis of 79 differentially expressed miRNAs (FDR<0.05). The disease status of the samples is shown at the top. Each column represents a miRNA and each row a sample. (The display indicates the logarithm of the expression changes, where varying shades of darker and lighter (red and green in original colour figure) indicate up and down-regulation, respectively).

FIG. 2: Validation of the four potential miRNAs biomarkers using RT-PCR. The number of samples analysed in each group is indicated in brackets. Significant adjusted p-values (P) are shown.

FIG. 3: ROC curves for individual miRNAs, miRNA-143 and miRNA-30e, and their combination.

FIG. 4: Urine concentration of the candidate protein biomarkers. A, Scatter dot plots of LYVE1, REG1A, REG1B and TFF1 protein concentration (creatinine-normalised) analysed by ELISA in healthy, chronic pancreatitis (CP) and pancreatic adenocarcinoma (PDAC) patients' urine. Upper bars: Kruskal-Wallis/Dunn's post test, ***: P<0.001; B, Statistical summary, median and Interquartile range (IQR) of raw/creatinine-normalised data for the biomarkers, median and IQR of urine creatinine (mmol/L), as well as plasma CA19.9 by sample groups are shown.

FIG. 5—Diagnostic performance of urine biomarkers in discriminating pancreatic adenocarcinoma patients from healthy controls. A, ROC curves of PDAC (n=143) versus healthy (n=59) subjects for individual creatinine-normalised urine biomarkers in the training set (70% of the data); B, ROC curves of PDAC versus healthy for the panel in the training set and in the independent validation set (30% of the data: PDAC n=49, healthy n=28); C, Summary table. AUC: area under the curve, SN: sensitivity, SP: specificity, with corresponding 95% Confidence Intervals (CI). SN and SP in the validation set are derived for optimal cut point determined in the training dataset. cnorm: creatinine-normalised, creat: creatinine.

FIG. 6—Urine concentration of the three biomarkers in different stages of pancreatic adenocarcinoma. Scatter dot plots of urine LYVE1, REG1A, TFF1 protein concentration (creatinine-normalised) in urines of healthy (n=87) and pancreatic adenocarcinoma patients at different stages of disease development (I-IIA n=16, I-II n=71, III-IV n=77). Bars indicate median and IQR values. Upper bars: Kruskal-Wallis/Dunn's post test, *: P<0.05, **: P<0.01, ***: P<0.001.

FIG. 7—Diagnostic performance of urine biomarkers in discriminating early pancreatic adenocarcinoma patients form healthy individuals. A, ROC curves of stages I-II PDAC (n=56) versus healthy (n=61) subjects for individual urine biomarkers in the training set (70% of the data); B, ROC curves of stage I-II PDAC versus healthy for the panel in the training set and in the independent validation set (30% of the data; PDAC n=15, healthy n=26); C, Summary table. AUC: area under the curve, SN: sensitivity, SP: specificity, with corresponding 95% Confidence Intervals (CI). SN and SP in the validation set are derived for optimal cutpoint determined in the training dataset. cnorm: creatinine-normalised, creat: creatinine.

FIG. 8—Diagnostic performance of the urine biomarker panel and CA19.9 in discriminating early pancreatic adenocarcinoma patients form healthy individuals. A, ROC curves of the biomarker panel with corresponding plasma CA19.9 alone and in combination comparing healthy urine (n=28), and urines from PDAC stages I-II, n=71 and I-IIA, n=16 (B). C, Summary table. AUC: area under the curve, SN: sensitivity, SP: specificity with 95% Confidence Interval (CI). SN and SP in the validation set were derived for optimal cutpoint determined in the training dataset.

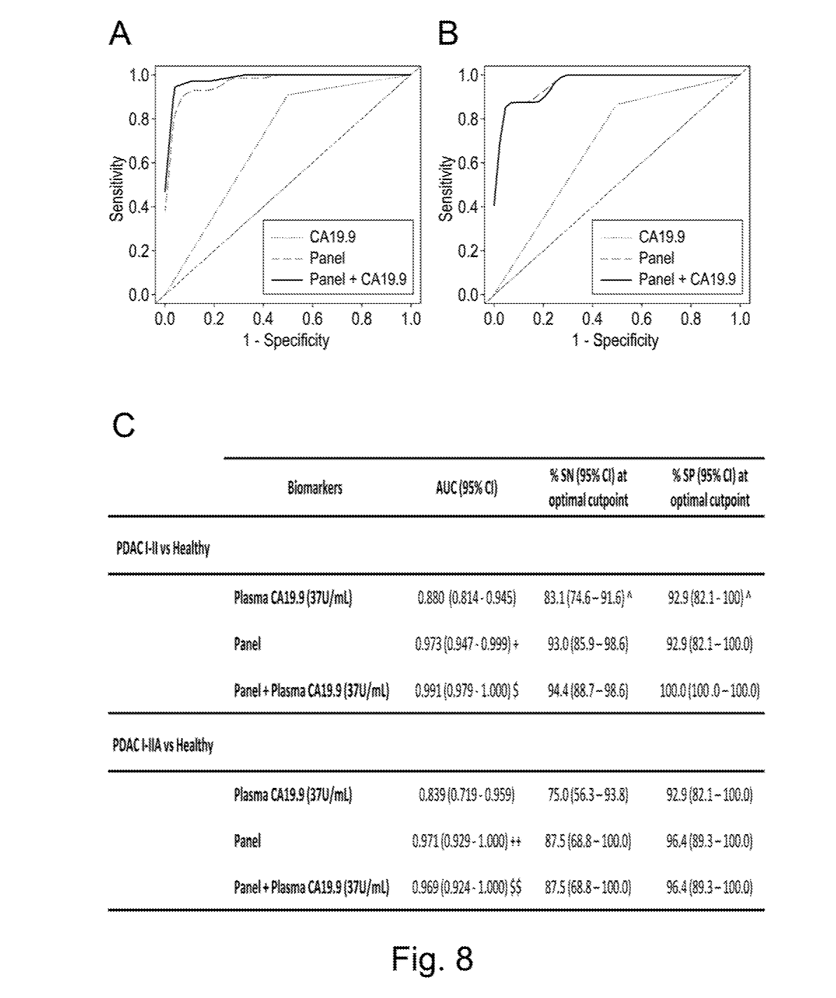

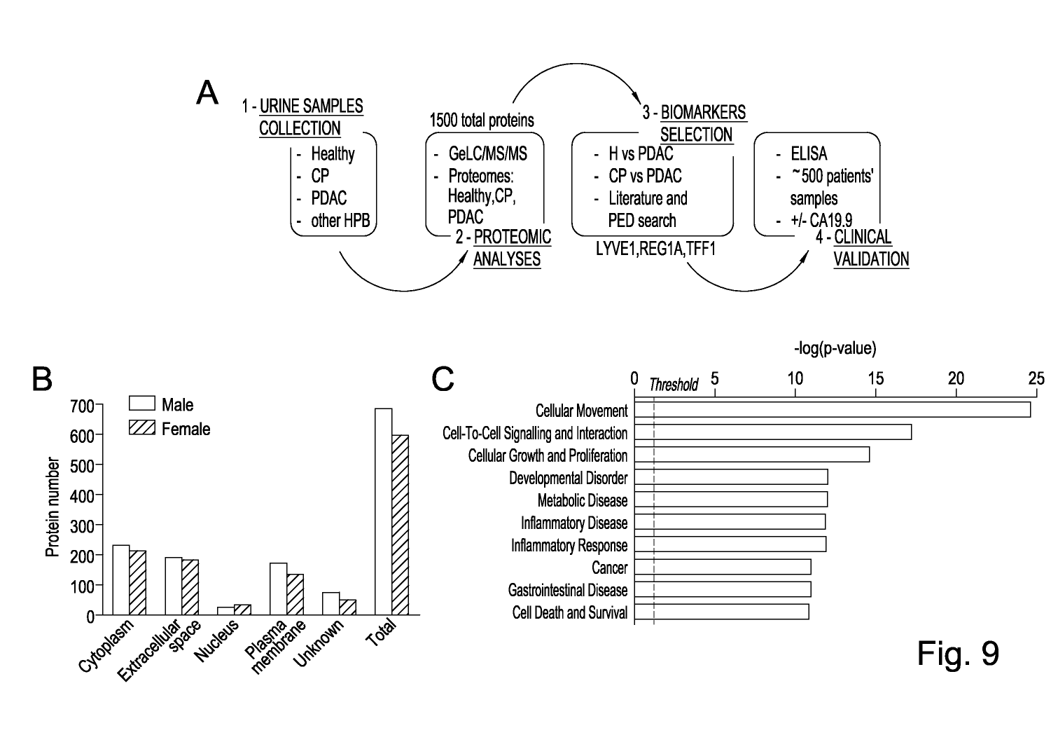

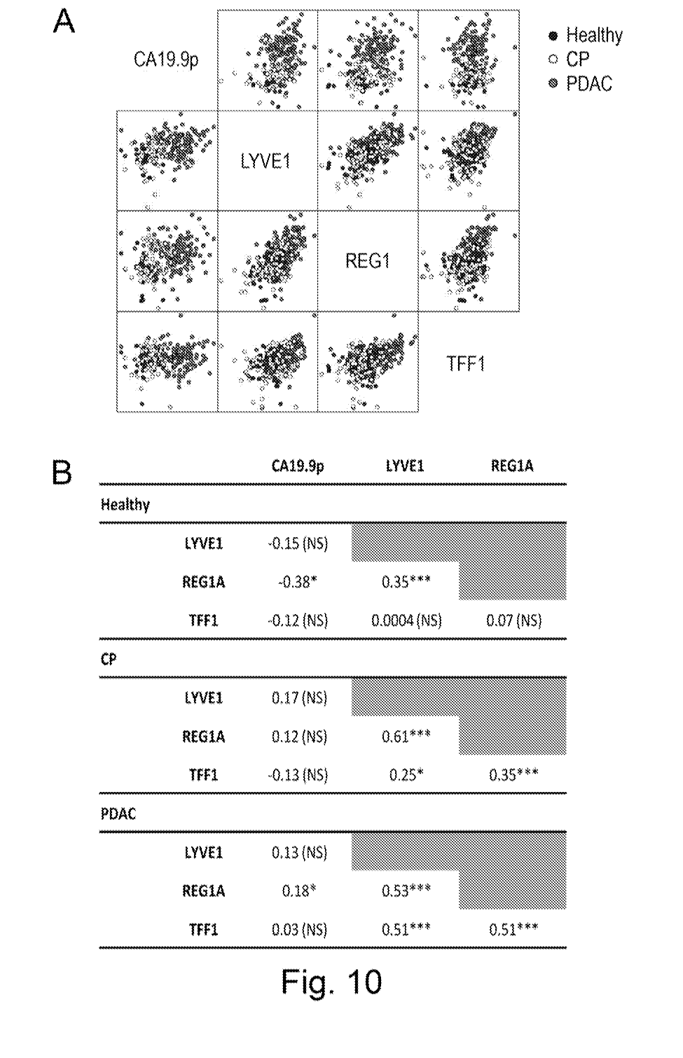

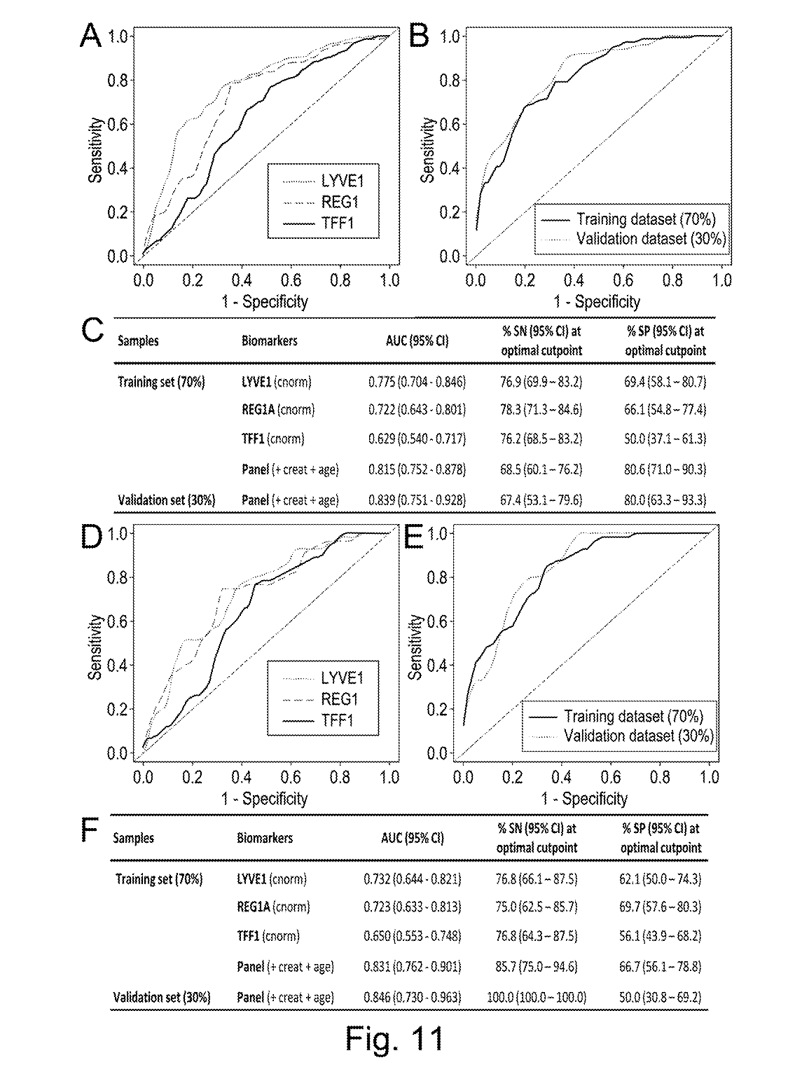

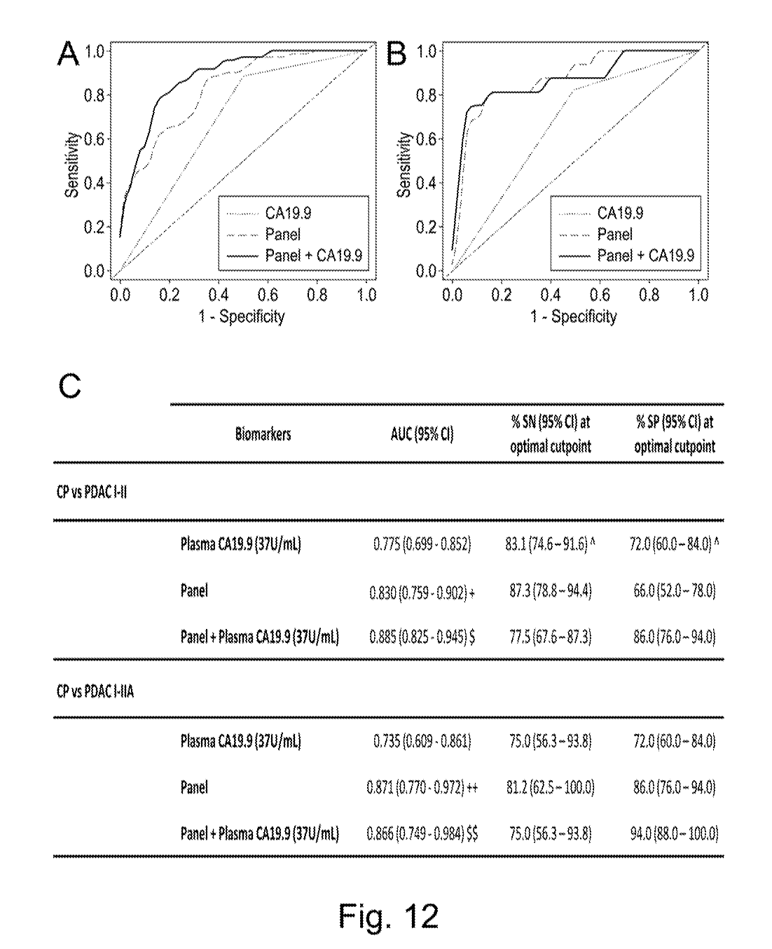

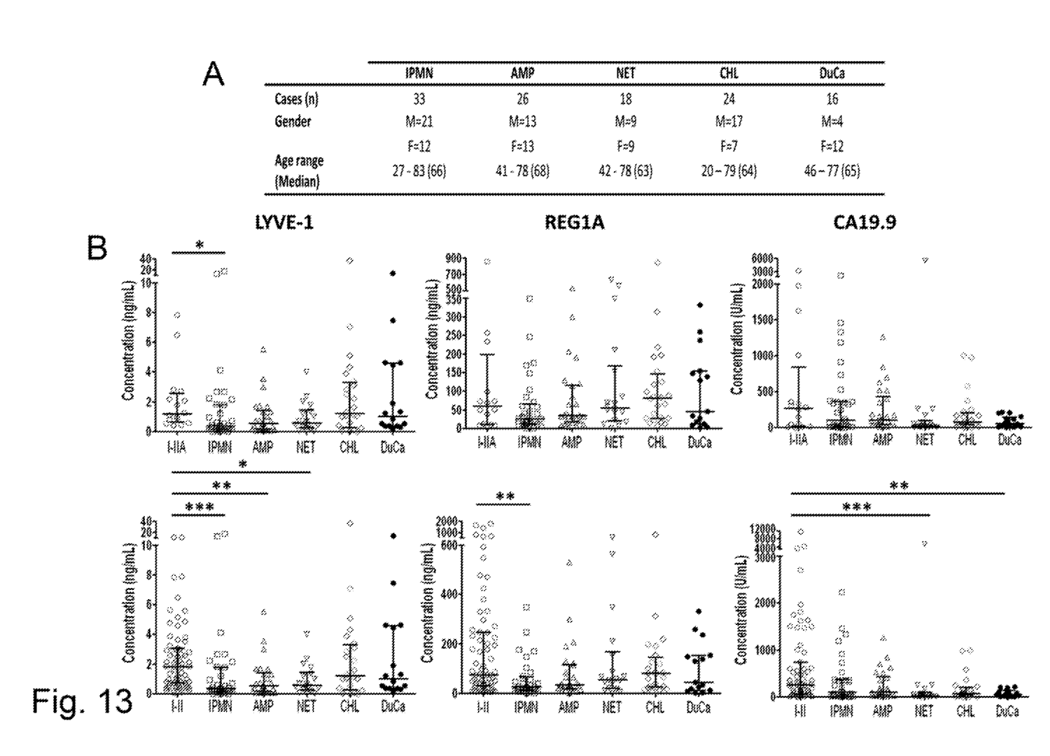

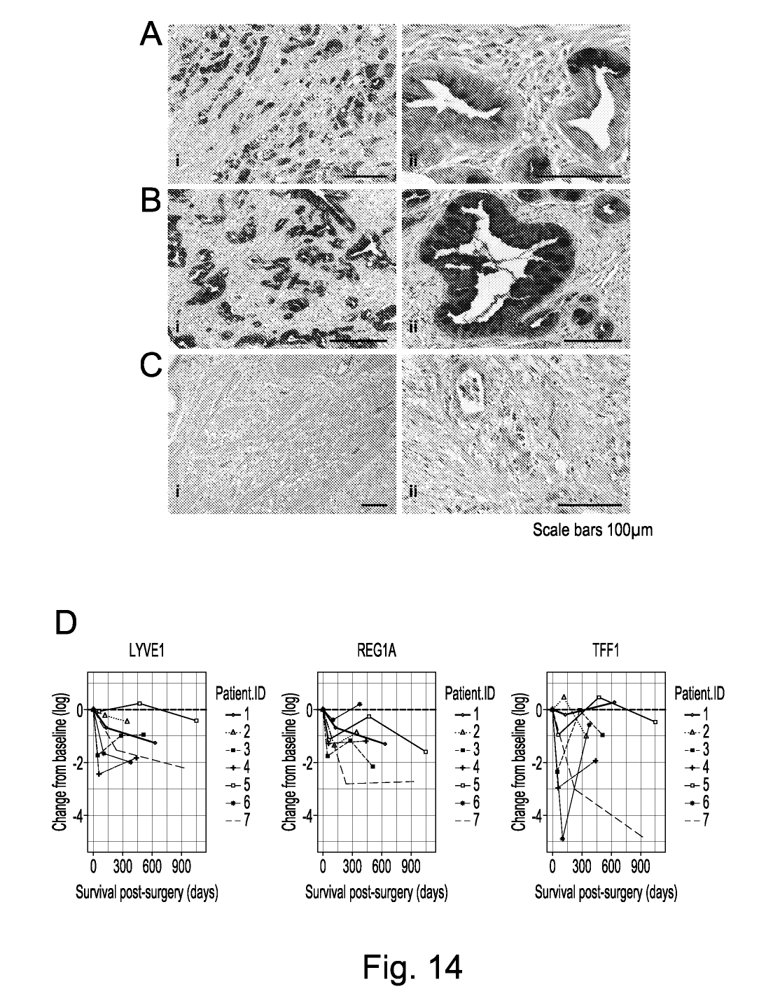

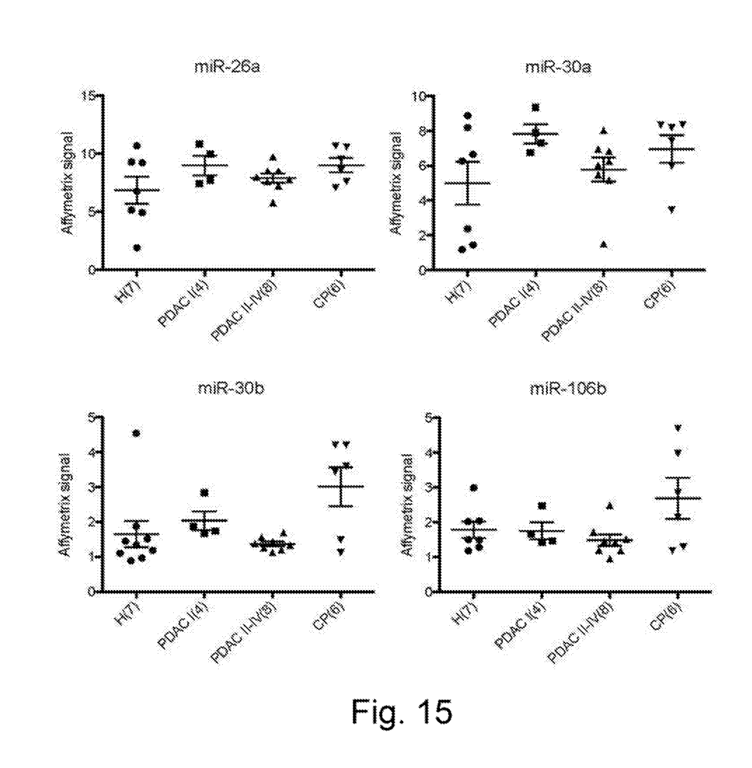

Legend for FIG. 8C

̂Optimal cutpoint for CA19.9 is 37 U/mL

+ DeLong's 1-sided test for correlated/paired AUCs to assess whether the urine panel gives a significantly greater AUC compared to plasma CA19.9 alone used as a dichotomous biomarker (0.973 versus 0.880), p=0.005

$ DeLong's 1-sided test for correlated/paired AUCs to assess whether the addition of plasma CA19.9 used as a dichotomous biomarker significantly increase the AUC over the urine panel alone (0.991 versus 0.973), p=0.04

++DeLong's 1-sided test for correlated/paired AUCs to assess whether the urine panel gives a significantly greater AUC compared to plasma CA19.9 alone used as a dichotomous biomarker (0.971 versus 0.839), p=0.006

$$ DeLong's 1-sided test for correlated/paired AUCs to assess whether the addition of plasma CA19.9 used as a dichotomous biomarker significantly increase the AUC over the urine panel alone (0.969 versus 0.971), p=0.7

FIG. 9—Urine proteome analysis. A, schematic outline of the study; B, classification of total identified proteins according to sub-cellular localisation; and C, functional activity determined by Ingenuity Pathway Analysis. H: healthy, CP: chronic pancreatitis, PDAC: pancreatic ductal adenocarcinoma, GeLC/MS/MS: SDS-PAGE-Liquid Chromatography-Tandem Mass Spectrometry.

FIG. 10—Correlation of the three urinary biomarkers and plasma CA19.9 (CA19.9p). A, Correlation plots (Navy blue/darkest: Healthy; Turquoise/lightest: chronic pancreatitis (CP); Purple/intermediate: pancreatic adenocarcinoma (PDAC). B, Pearson correlation coefficients and corresponding significance (NS: non-significant, *: P<0.05, **: P<0.01, ***: P<0.001).

FIG. 11—Diagnostic performance of urine biomarkers in discriminating pancreatic adenocarcinoma all stages (A-C) and stage I-II (D-F) from chronic pancreatitis patients. A, ROC curves of PDAC (n=143) versus CP (n=62) patients for individual urine biomarkers in the training set (70% of the data). B, ROC curves of PDAC versus CP patients for the panel in the training set and in the independent validation set (30% of the data, PDAC n=49, CP n=30). C, Summary table. D, ROC curves of individual urine biomarkers in training dataset (70%, PDAC n=56, CP=66). E, ROC curves of the panel in training and validation (PDAC n=15, CP n=26) dataset. F, Summary table. Cnorm, creatinine-normalised, creat, creatinine, AUC: area under the curve SN: sensitivity, SP: specificity. with 95% Confidence Interval (CI). SN and SP in the validation set were derived for optimal cutpoint determined in the training dataset.

FIG. 12—Exploratory comparison of plasma CA19.9 and the urine biomarker panel in discriminating early pancreatic adenocarcinoma from chronic pancreatitis patients. A, ROC curves of the biomarker panel with corresponding plasma CA19.9 alone and in combination comparing CP urine (n=50), and urines from PDAC stages I-II (n=71) and I-IIA (n=16) (B). C, Summary table. AUC: area under the curve, SN: sensitivity, SP: specificity with 95% Confidence Interval (CI). SN and SP in the validation set were derived for optimal cutpoint determined in the training dataset.

Legend for FIG. 12C:

̂Optimal cutpoint for CA19.9 is 37 U/mL

+ DeLong's 1-sided test for correlated/paired AUCs to assess whether the urine panel gives a significantly greater AUC compared to plasma CA19.9 alone used as a dichotomous biomarker (0.830 versus 0.775), p=0.1

$ DeLong's 1-sided test for correlated/paired AUCs to assess whether the addition of plasma CA19.9 used as a dichotomous biomarker significantly increase the AUC over the urine panel alone (0.885 versus 0.830), p=0.01

++ DeLong's 1-sided test for correlated/paired AUCs to assess whether the urine panel gives a significantly greater AUC compared to plasma CA19.9 alone used as a dichotomous biomarker (0.871 versus 0.735), p=0.004

$$ DeLong's 1-sided test for correlated/paired AUCs to assess whether the addition of plasma CA19.9 used as a dichotomous biomarker significantly increase the AUC over the urine panel alone (0.866 versus 0.871), p=0.6

FIG. 13—Urine biomarker concentrations in different tumours. A, Demographic details. B, Scatter dot plots of urine LYVE1, REG1A and plasma CA19.9 in different hepatobiliary pathologies and early stages of pancreatic adenocarcinoma (I-IIA, n=16) and I-II (n=71). The level of TFF1 protein was not measured in these samples due to substantial modifications made to the original ELISA assay by the source company at the moment of this analysis. IPMN (n=33): intraductal papillary mucinous neoplasm, AMP (n=26): ampullary cancer, NET (n=18): neuroendocrine tumour, CHL (n=24): cholangiocarcinoma, DuCA (n=16): duodenal cancer. Bars indicate median and IQR values. Upper bars: Kruskal-Wallis/Dunn's post test, *: P<0.5, **: P<0.01, ***: P<0.001; where not shown, difference not statistically significant.

FIG. 14—Expression of the biomarker panel proteins in pancreatic cancer tissues. A, Immunohistochemical analysis of REG1A: i) REG1A in poorly differentiated PDAC, ii) luminal REG1A in malignant glands. B, TFF1: i) heterogenous expression in cancer, ii) luminal TFF1 in malignant gland. C, LYVE1 expression in the scattered lymphatic vessels i) in the muscle layer and ii) in the stroma surrounding malignant gland. D, The biomarker levels during monitoring of pancreatic adenocarcinoma patients: LYVE1, REG1A and TFF1 were measured using ELISA in urine samples collected before surgery and during the patients' follow up. Each point represents log-transformed ELISA values at a particular time point (x-axis).

FIG. 15—Differential diagnosis of chronic pancreatitis (CP) versus healthy, versus stage I PDAC, and versus state II-IV PDAC

Affymetrix data analysis showing the significant differential expression of four additional miRNAs in chronic pancreatitis (CP): miR-26a, miR-30a, miR-30b, and miR-106b. All four miRNAs were over-expressed in CP when compared to healthy individuals. Furthermore, miR-30b and miR-106b were elevated in CP when compared with pancreatic ductal adenocarcinoma (PDAC).

### DETAILED DESCRIPTION OF THE INVENTION

### Biomarker Panels

The present invention provides a biomarker panel useful in methods of diagnosis and treatment of pancreatic ductal adenocarcinoma (PDAC). In one embodiment, the panel comprises miR-143, miR-223, miR-204 and/or miR-30e. In particular, the present invention provides a method of diagnosing, screening or testing for, and methods of treating or preventing PDAC, comprising detecting or quantifying the level of expression of an miRNA selected from the group consisting of miR-143, miR-223, miR-204 and miR-30e, or combinations thereof, in a biological sample.

MicroRNAs (miRNAs) are short nucleotides of RNA (20 to 25 nucleotides, such as 22 nucleotides, in length) that are important in gene regulation, a function that is effected by binding of the miRNAs to mRNA. They are found in a number of tissues, including pancreatic tissue, as well as blood and urine. The miRNAs of interest in the present invention are human miRNAs, and so the present invention is particularly useful for treatment and diagnosis in humans. However, since miRNAs are highly conserved, the present invention may equally be useful in other animals, such as mammals in general.

The full name for the mature sequence of miR-143 is hsa-miR-143-3p (Accession number MIMAT0000435 in miRBase, release 21, June 2014), and the sequence is as follows:

(SEQ ID NO: 5)
UGAGAUGAAGCACUGUAGCUC

The full name for the mature sequence of miR-223 is hsa-miR-223-3p (Accession number MIMAT0000280 in miRBase, release 21, June 2014),

(SEQ ID NO: 6)
UGUCAGUUUGUCAAAUACCCCA

The full name for the mature sequence of miR-30e is hsa-miR-30e-5p (Accession number MIMAT0000692 in miRBase, release 21, June 2014), and the sequence is as follows:

(SEQ ID NO: 7)
UGUAAACAUCCUUGACUGGAAG

The full name for the mature sequence of miR-204 is hsa-miR-204-5p (Accession number MIMAT0000265 in miRBase, release 21, June 2014), and the sequence is as follows:

(SEQ ID NO: 8)
UUCCCUUUGUCAUCCUAUGCCU

The full name for the mature sequence of miR-26a is hsa-miR-26a-5p (Accession number MIMAT0000082 in miRBase, release 21, June 2014), and the sequence is as follows:

(SEQ ID NO: 9)
UUCAAGUAAUCCAGGAUAGGCU

The full name for the mature sequence of miR-30a is hsa-miR-30a-5p (Accession number MIMAT0000420 in miRBase, release 21, June 2014), and the sequence is as follows:

(SEQ ID NO: 10)
UGUAAACAUCCUCGACUGGAAG

The full name for the mature sequence of miR-30b is hsa-miR-30a-5p (Accession number MIMAT0000087 in miRBase, release 21, June 2014), and the sequence is as follows:

(SEQ ID NO: 11)
UGUAAACAUCCUACACUCAGCU

The full name for the mature sequence of miR-106b is hsa-miR-106b-5p (Accession number MIMAT0000680 in miRBase, release 21, June 2014), and the sequence is as follows:

(SEQ ID NO: 12)
UAAAGUGCUGACAGUGCAGAU

In the present invention, an increase in expression (i.e. over-expression) compared to a healthy patient in one or all of miR-143, miR-223, miR-204, miR-30e, miR-26a, miR-30a, miR-30b and miR-106b indicative of PDAC or CP. False negatives and false positives can also be minimised by utilising a combination of panel members together. “Healthy” patients is intended to refer to patients without PDAC (of any severity), without CP. In some embodiments, the healthy patients will not have IPMNs or other cystic lesions.

Any biomarkers or combination of biomarkers of the invention can be used in any of the methods, uses, kits etc. described herein. However, the specific choice of biomarkers analysed may depend on the context. For example, miR-143 is particularly useful in distinguishing between stage I PDAC and healthy patients. Results are improved even further when miR-143 is combined with miR-30e. These two miRNAs may be further combined with miR-223. Therefore, these miRNAs or combinations of miRNAs may be preferred in methods, uses, kits etc. that relate to diagnosis or treatment of PDAC, in particular early stage PDAC.

Alternatively, a panel comprising miR-143, miR-223 and miR-204 is particularly good at distinguishing between stage I and later stages of PDAC. Therefore, these miRNAs or combinations of miRNAs may be preferred in methods, uses, kits etc. that relate to diagnosis or treatment of PDAC, in particular late stage PDAC, and the differential diagnosis of late stage PDAC and early stage PDAC.

Furthermore, a panel comprising miR-223 and miR-204 is particularly good at distinguishing between early stage PDAC and chronic pancreatitis. The biomarkers miR-30b and miR-106b are also good at distinguishing between early stage PDAC and chronic pancreatitis. Therefore, these miRNAs or combinations of miRNAs may be preferred in methods, uses, kits etc. that relate to the diagnosis or treatment of early stage PDAC or CP, and the differential diagnosis of early stage PDAC and CP.

In addition, the biomarkers miR-26a, miR-30a, miR-30b and miR-106b are each able to distinguish between chronic pancreatitis and healthy patients. Therefore, these miRNAs or combinations of miRNAs may be preferred in methods, uses, kits etc. that relate to the diagnosis or treatment of CP.

Further, the biomarkers miR-30b and miR-106b are additionally able to distinguish between CP and stage 1 PDAC (as noted above), and between CP and stage II-IV PDAC. Therefore, these miRNAs or combinations of miRNAs may be preferred in methods, uses, kits etc. that relate to the diagnosis or treatment of any of CP, early stage PDAC and/or late stage PDAC, as well as in the differential diagnosis between CP and PDAC, in particular between CP and stage I PDAC and between CP and stage II-IV PDAC.

Further still, each of the biomarkers miR-26a, miR-30a, miR-30b and miR-106b are able to predict the progression of pancreatic disease from cysts (such as IPMNs) to malignant PDAC. Therefore, these miRNAs or combinations therefore may be preferred in methods, uses, kits etc. that relate to the prognosis of patients having or at risk of developing malignant PDAC. In such embodiments, the biomarkers are from a urine sample.

According, certain biomarkers and combinations of biomarkers are useful in the classification of pancreatic disease.

The selection of biomarkers and design of biomarker panels and kits can be designed accordingly.

In some embodiments of the invention, the miRNA biomarker(s) can be combined with a protein biomarker panel. In such embodiments, the invention the method may comprise detecting the level of expression or concentration of a protein selected from the group consisting of LYVE1, REG1 and TFF1, or combinations thereof, in a urine sample. In some embodiments of the invention, the method may comprise quantifying the level of expression or concentration of only one of LYVE1, REG1 and TFF1 in a urine sample. In other embodiments of the invention, the method may comprise quantifying the level of expression or concentration of any two of LYVE1, REG1 and TFF1, for example LYVE1 and REG1 (REG1A and/or REG1B), LYVE1 and TFF1, or REG1 (REG1A and/or REG1B) and TFF1. In some embodiments of the invention, the method may comprise detecting the level of expression of all of LYVE1, REG1 (REG1A and/or REG1B) and TFF1. CA19.9 may also be quantified in some embodiments of the invention.

LYVE1 is also known as lymphatic vessel endothelial hyaluronan receptor and extracellular link domain containing 1 (XLKD1) is a type I integral membrane glycoprotein. The encoded protein acts as a receptor and binds to both soluble and immobilized hyaluronan. References to LYVE include NCBI (GenBank) reference sequence transcript NM_006691.3 (GI:15130120), NCBI protein ID NP_006682.2 (GI:40549451), GeneID:10894, and HGNC (HUGO (Human Gene Organisation) Gene Nomenclature Committee gene ID):14687. The gene has 4 splice variants; two are protein coding (full length protein has 322 amino acids, and the second one 218 amino acids). The full sequence of the protein is as follows:

(SEQ ID NO: 1)
MARCFSLVLLLTSIWTTRLLVQGSLRAEELSIQVSCRIMGITLVSKK
ANQQLNFTEAKEACRLLGLSLAGKDQVETALKASFETCSYGWVGDGF
VVISRISPNPKCGKNGVGVLIWKVPVSRQFAAYCYNSSDTWTNSCIP
EIITTKDPIFNTQTATQTTEFIVSDSTYSVASPYSTIPAPTTTPPAP
ASTSIPRRKKLICVTEVFMETSTMSTETEPFVENKAAFKNEAAGFGG
VPTALLVLALLFFGAAAGLGFCYVKRYVKAFPFTNKNQQKEMIETKV
VKEEKANDSNPNEESKKTDKNPEESKSPSKTTVRCLEAEV

REG1A refers to regenerating islet-derived protein 1 that belongs to a family of REG (regenerating) glycoproteins, which are expressed in pancreatic acinar cells and act as both autocrine and paracrine growth factors. The Reg gene family is a multigene family grouped into four subclasses, types I, II, III and IV; REG1A gene is a type I subclass member. Other family members: REG1B, REGL, PAP and this gene are tandemly clustered on chromosome 2p12 and may have arisen from the same ancestral gene by gene duplication. REG1A encodes a protein that is secreted by the exocrine pancreas. References to REG1 herein include two commonly described genes, i.e. REG1A and REG1B, whose products are more than 80% identical at the protein level, and are difficult to distinguish. Hence in the present invention, the method may quantify the expression or concentration of just one of REG1A and REG1B. In alternative embodiments of the invention, the expression or concentration of both REG1A and REG1B may be separately quantified. In a further embodiment of the invention, a quantification method may be used in which REG1A and REG1B cannot be distinguished and hence they may be quantified together.

References to REG1A include NCBI (GenBank) reference sequence transcript NM_002909.4 (GI:189491780), NCBI protein ID NP_002900.2 (GI:29725633), GeneID:5967 and HGNC:9951. This gene has 4 splice variants (retained introns), but only one is protein coding (166 amino acids).

(SEQ ID NO: 2)
MAQTSSYFMLISCLMFLSQSQGQEAQTELPQARISCPEGTNAYRSYC
YYFNEDRETWVDADLYCQNMNSGNLVSVLTQAEGAFVASLIKESGTD
DFNVWIGLHDPKKNRRWHWSSGSLVSYKSWGIGAPSSVNPGYCVSLT
SSTGFQKWKDVPCEDKFSFVCKFKN

References to REG1B include NCBI (GenBank) reference sequence transcript NM_006507.3 (GI:189491779), NCBI protein ID NP_006498.1, GeneID:5968 and HGNC:9952. There are 5 splice variants of this gene (retained introns), only two code for the proteins of 166 amino acids and 149 amino acids.

(SEQ ID NO: 3)
MAQTNSFFMLISSLMFLSLSQGQESQTELPNPRISCPEGTNAYRSYC
YYFNEDPETWVDADLYCQNMNSGNLVSVLTQAEGAFVASLIKESSTD
DSNVWIGLHDPKKNRRWHWSSGSLVSYKSWDTGSPSSANAGYCASLT
SCSGFKKWKDESCEKKFSFVCKFKN

TFF1 refers to trefoil factor 1. TFF1 belongs to a family of gastrointestinal secretory peptides, which interact with mucins and are expressed at increased levels during reconstitution and repair of mucosal injury. They protect epithelial cells from apoptotic death and increase their motility, but also play similar pivotal roles in cancer cells, and are thus involved in the development and progression of various cancer types. References to TFF1 include NCBI (GenBank) reference sequence transcript NM_003225.2 (GI:48928023), NCBI protein NP_003216.1 (GI:4507451), Gene ID:7031 and HGNC:11755.

(SEQ ID NO: 4)
MATMENKVICALVLVSMLALGTLAEAQTETCTVAPRERQNCGFPGVT
PSQCANKGCCFDDTVRGVPWCFYPNTIDVPPEEECEF

The method of the invention can be performed in a qualitative format, which determines the presence or absence of a cancer biomarker in the sample, but preferably will be done in a quantitative format, which, in addition, provides a measurement of the quantity of cancer marker present in the sample. The quantity of marker present in the sample may be calculated using any of techniques described herein, or others known by the skilled person to be suitable. Prior to performing a quantitative assay, it may be necessary to draw a standard curve by measuring the signal obtained using the same detection reaction that will be used for the assay from a series of standard samples containing known concentrations of the biomarker. The quantity of biomarker present in a sample to be screened can then extrapolated from the standard curve.

Generally, an increase in one or more of the biomarkers in a test sample compared to a control sample from a healthy patient indicates the presence of chronic pancreatitis and/or PDAC. The threshold concentrations of each of the biomarkers may differ from patient to patient or population to population. The relative quantification of miRNA expression in PDAC compared to healthy patient or those with CP allow a diagnosis to be made.

Indicative protein concentrations from unprocessed (“raw” or “crude”) urine samples are shown below:

| | Healthy | CP | PDAC |
|---|---|---|---|
| LYVE1 | ≤2 ng/ml | 2-10 ng/ml | ≥10 ng/ml |
| REG1A | ≤120 ng/ml | 120-500 ng/ml | ≥500 ng/ml |
| REG1B | ≤40 ng/ml | 40-100 ng/ml | ≥100 ng/ml |
| TFF1 | ≤2.5 ng/ml | 2.5-4 ng/ml | ≥4 ng/ml |

For example, if a sample contains between 2 and 10 ng/ml LYVE1, between 120 and 500 ng/ml REG1A, between 40 and 100 ng/ml REG1B and/or between 2.5 to 5 ng/ml TFF1, chronic pancreatitis may be suspected.

However, the above ranges are indicative, and the skilled person will realise that the concentration of each biomarker will need to be considered in context, for example depending on the origin of the sample and any pre-processing of that sample that may have taken place. The more of the biomarkers that fall within the relevant concentration thresholds, the more likely it is the patient is healthy, has CP or has PDAC, as the case may be.

Of course, methods of diagnosis using the biomarker panels of the invention can be further confirmed by, for example, testing a biopsy for the presence of PDAC.

### Types of Pancreatic Cancer

The methods of the present invention are particularly useful with respect to early-stage PDAC. Hence, methods of treatment and diagnosis are useful in the treatment and diagnosis of early-stage PDAC, and methods of prevention are useful in the prevention of late-stage PDAC (such as stage II to stage IV PDAC, or stage III to stage IV PDAC).

In one embodiment of the invention there is thus provided a method of diagnosing or detecting stage I PDAC, comprising determining the level of expression or concentration of miR-143, miR-223, miR-204 and/or miR-30e in a urine sample and comparing each of the determined expression levels with a reference (or references). The methods in particular may determine the presence of stage I PDAC and distinguish these from healthy patients, patients having CP and potentially patients having IPMNs or other cystic lesions. miR-143 is particularly suited for detection of early stage (stage I) PDAC, especially when combined with miR-30e. However, miR-223 may additionally be used as the inventors have found that it is also over-expressed in stage I PDAC.

The methods and biomarker panels of the invention are useful to distinguish between PDAC (especially early stage PDAC) and CP. For example, the combination of miRNA biomarkers miR-223 and miR-204 are particularly good at distinguishing between PDAC and CP. miR-30b and/or miR106b are particularly useful in distinguishing between CP and stage I PDAC and between CP and stage II-IV PDAC.

The methods and biomarker panels of the invention are also useful to distinguish between stage I and later stages of PDAC. For example, the combination of the miRNA biomarkers miR-143, miR-223 and miR-204 are particularly good at distinguishing between early stage (stage I) PDAC and later stages (II-IV) PDAC.

Some methods and biomarker panels of the invention are useful to diagnose CP. For example, each of miR-26a, miR-30a, miR-30b and miR106b are particularly useful in distinguishing between CP and healthy patients.

Classification of PDAC can be done according to the The American Joint Committee on Cancer (AJCC) tumour-nodes-metastasis (TNM) staging system. The T score describes the size of the main (primary) tumour and whether it has grown outside the pancreas and into nearby organs. The N score describes the spread to nearby (regional) lymph nodes. The M score indicates whether the cancer has metastasized (spread) to other organs of the body:

Tx, T0, Tis: see TNM system
T1: tumour <2 cm in greatest dimension, limited to pancreas
T2: tumour >2 cm in greatest dimension, limited to pancreas
T3: extension beyond pancreas, no involvement of SMA or coeliac axis
T4: involvement of SMA or coeliac axis

Regional lymph nodes (N)

Nx: nodes cannot be assessed
N0: no evidence of nodal involvement
N1: regional nodal metastases present

Metastases (M)

Mx: presence of metastases cannot be assessed
M0: no evidence of metastases
M1: distant metastases present

Stage I PDAC is the earliest stage, where cancer is confined to the pancreas, and there is no cancer in the lymph nodes. In Stage II, the cancer is locally invasive. Cancer in both of these stages is still resectable; currently, fewer than 1 in 5 cancers of the pancreas (<20%) are diagnosed at stage I/11. References to stage II PDAC herein include stage IIA and IIB. In stage III, cancer has spread beyond pancreas and is in large blood vessels, so unresectable. Stage IV cancer has metastasized to distant sites (and again not treatable by surgery). References herein to detecting or diagnosing PDAC generally refer to detecting or diagnosing each stage PDAC, in particular stage I or stage II PDAC. Such methods are particularly useful given the cancer is still treatable by resection at this stage and survival rates are much improved.

With reference to the TNM score, the stage groupings are:
stage 0: Tis N0 M0
stage Ia: T1 N0 M0
stage Ib: T2 N0 M0
stage IIa: T3 N0 M0
stage IIb: T1, T2 or T3 with N1 M0
stage III: T4 and M0 (any N)
stage IV: M1 (any T any N)

References to Stage I PDAC herein including both stage Ia and stage Ib, unless otherwise stated.

### Biological Samples

In the present invention, the biological sample may be a urine sample, a whole blood sample, a saliva sample, a serum sample or a biopsy (such as a pancreatic tissue sample), although urine samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing within two hours of collection. Maintenance at −80° C. can be used for long-term storage.

The sample may be processed prior to determining the level of expression of the biomarkers. The sample may be subject to enrichment (for example to increase the concentration of the biomarkers being quantified), centrifugation or dilution. In other embodiments, the samples do not undergo any pre-processing and are used unprocessed (such as whole urine)

In some embodiments of the invention, the biological sample may be enriched for miRNA prior to detection and quantification (i.e. measurement). The step of enrichment can be any suitable pre-processing method step to increase the concentration of miRNA in the sample. For example, the step of enrichment may comprise centrifugation and/or filtration to remove cells or unwanted analytes from the sample. Methods of the invention may include a step of amplification to increase the amount of miRNA that is detected and quantified. Methods of amplification include PCR amplification. Such methods may be used to enrich the sample for any biomarkers of interest.

Generally speaking, the miRNAs will need to be extracted from the biological sample. This can be achieved by a number of suitable methods. For example, extraction may involve separating the miRNAs from the biological sample. Methods include chemical extraction (comprising the use of, for example, guanidium thiocyante) and solid-phase extraction (for example on silica columns). Preferred methods include chromatographic methods (for example spin column chromatography), in particular chromatographic methods comprising the use of a silica column. Chromatographic methods comprise lysing cells (if required), addition of a binding solution, centrifugation in a spin column to force the binding solution through a silica gel membrane, optional washing to remove further impurities, and elution of the nucleic acid. Commercial kits are available for such methods, for example Norgen's urine microRNA purification kit (other kits available, for example from Qiagen or Exigon).

If miRNAs are extracted from a sample, the extracted solution may require enrichment to increase the relative abundance of miRNAs in the sample.

The methods of the invention may be carried out on one test sample from a patient. Alternatively, a plurality of test samples may be taken from a patient, for example at least 2, at least 3, at least 4 or at least 5 samples. Each sample may be subjected to a single assay to quantify one of the biomarker panel members, or alternatively a sample may be tested for all of the biomarkers being quantified.

### Methods of the Invention

In one embodiment of the invention, the method comprises the steps of:
a) detecting biomarkers of interest, in particular miRNAs, in a biological sample obtained from a patient; and
b) quantifying the expression levels of each of the biomarkers (such as miRNA molecules).

The biomarkers belong to the biomarker panels of the invention. Hence, detection/quantification comprises detection/quantification of one or more of the following biomarkers:
1) miR-143
2) miR-223
3) miR-204
4) miR-30e
5) miR-26a
6) miR-30a
7) miR-30b
8) miR-106b
9) LYVE1
10) REG1 (REG1A and/or REG1B)
11) TFF1
12) CA19.9

Preferably, the invention comprises analysis of at least one biomarker selected from the group consisting of miR-143, miR-223, miR-204 and miR-30e, at least one biomarker selected from the group consisting of LYVE1, REG1 and TFF1, and/or the biomarker CA19.9.

For example, the biomarker panel may comprise miR-143 and CA19.9 or miR-143, miR-30e and CA19.9. In one embodiment, the panel comprises miR-143, miR-30e and one of either miR-223 or miR-204, optionally further comprising CA19.9.

In some embodiments, in particular those relating to CP and distinguishing between CP and PDAC, the biomarker panel may comprise miR-26a, miR-30a, miR-30b and/or miR-106b.

Regarding miRNA biomarkers, the step of detection may comprise a detection method based on hybridisation, amplification or sequencing, or cellular phenotypic change, or the detection of binding of a specific molecule, or a combination thereof. Methods based on hybridisation include Northern blot, microarray, solid phase detection, NanoString, RNA-FISH, branched chain hybridisation assay analysis, and related methods. Methods based on amplification include quantitative reverse transcription polymerase chain reaction (qRT-PCT) and transcription mediated amplification, and related methods. Methods based on sequencing include Sanger sequencing, next generation sequencing (high throughput sequencing by synthesis) and targeted RNAseq, nanopore mediated sequencing (MinION), Methods based on phenotypic change may detect changes in test cells or in animals as per methods used for screening miRNAs (for example, see Cullen & Arndt, Immunol. Cell Biol., 2005, 83:217-23). Methods based on binding of specific molecules include detection of binding to, for example, antibodies or other binding molecules such as RNA or DNA binding molecules or LNA (locked nucleic acid) approach. Methods based on enzymatic digestion, for example the non-PCR MARS (MicroRNA-RNase-SPR) assay to detect specific miRNAs from human subjects by surface plasmon resonance (SPR) may also be used (J. F. C. Loo, et al., Analyst, 2015; 140: 4566-4575).

In the present invention, the solid phase detection, or liquid-phase hybridisation detection, may be used (the latter is more efficient), although any suitable technique can be used.

In methods of the invention, the step of detection and quantification may occur simultaneously. In such embodiments, there is no need for a separate detection step prior to quantification.

The level of expression of a biomarker can be quantified in a number of ways. Levels of expression may be determined by, for example, quantifying the biomarkers by determining the concentration of miRNA in the sample (such as a urine sample). Methods include real-time quantitative PCR, microarray analysis, RNA sequencing, Northern blot analysis and in situ hybridisation. There is also an nCounter Analysis system from NanoString and ‘Integrated Comprehensive Droplet Digital Detection’ (IC 3D) that has been developed for the digital quantification of miRNA directly in plasma (K. Zhang, et al., Lab on a Chip, first published online 14 Sep. 2015; DOI: 10.1039/C5LC00650C). In this system the plasma sample containing target miRNAs is encapsulated into microdroplets, enzymatically amplified and digitally counted using a novel, high-throughput 3D particle counter.

Methods of real-time qPCR can use stem-loop primers or a poly(A)tailing technique, to reverse transcribe RNA into complementary DNA (cDNA) for the amplification step. Generally using pre-designed assays that target specific miRNAs of interest. Microarray analysis may comprise the steps of fluorescently labelling the miRNAs, hybridization of the labelled miRNAs to DNA (or RNA or LNA) probes on a solid-substrate array, washing the array, and scanning the array. miRNA enrichment techniques may be particularly useful in methods involving microarrays.

RNA sequencing is another method that can benefit from miRNA enrichment, although this is not always necessary. RNA sequencing techniques generally using next generation sequencing methods (also known as high-throughput or massively parallel sequencing). These methods use a sequencing-by-synthesis approach and allow relative quantification and precise identification of miRNA sequences. In situ hybridisation techniques can be used on tissue samples, both in vivo and ex vivo.

In some methods of the invention, detection and quantification of cDNA-binding molecule complexes may be used to determine miRNA expression. For example, miRNA transcripts in a sample may be converted to cDNA by reverse-transcription, after which the sample is contacted with binding molecules specific for the miRNAs being quantified, detecting the presence of a of cDNA-specific binding molecule complex, and quantifying the expression of the corresponding gene. There is therefore provided the use of cDNA transcripts corresponding to one or more of the miRNAs of interest, or combinations thereof, for use in methods of detecting, diagnosing or prognosis PDAC, in particular early stage PDAC. In some embodiments of the invention, the method may therefore comprise a step of conversion of the miRNAs to cDNA to allow a particular analysis to be undertaken and to achieve miRNA quantification.

Methods for detecting the levels of protein expression include any methods known in the art. For example, protein levels can be measured indirectly using DNA or mRNA arrays. Alternatively, protein levels can be measured directly by measuring the level of protein synthesis or measuring protein concentration.

DNA and RNA arrays (microarrays) for use in quantification of the miRNAs of interest comprise a series of microscopic spots of DNA or RNA oligonucleotides, each with a unique sequence of nucleotides that are able to bind complementary nucleic acid molecules. In this way the oligonucleotides are used as probes to which only the correct target sequence will hybridise under high-stringency condition. In the present invention, the target sequence can be the coding DNA sequence or unique section thereof, corresponding to the miRNA whose expression is being detected. Most commonly the target sequence is the miRNA biomarker of interest itself.

Protein microarrays can also be used to directly detect protein expression. These are similar to DNA and RNA microarrays in that they comprise capture molecules fixed to a solid surface.

Capture molecules include antibodies, proteins, aptamers, nucleic acids, receptors and enzymes, which might be preferable if commercial antibodies are not available for the analyte being detected. Capture molecules for use on the arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. The appropriate capture molecule will depend on the nature of the target (e.g. mRNA, protein or cDNA).

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltametry and amperometry methods) and radio frequency methods (for example multipolar resonance spectroscopy).

With respect to protein biomarkers, direct measurement of protein expression and identification of the proteins being expressed in a given sample can be done by any one of a number of methods known in the art. For example, 2-dimensional polyacrylamide gel electrophoresis (2D-PAGE) has traditionally been the tool of choice to resolve complex protein mixtures and to detect differences in protein expression patterns between normal and diseased tissue. Differentially expressed proteins observed between normal and tumour samples are separate by 2D-PAGE and detected by protein staining and differential pattern analysis. Alternatively, 2-dimensional difference gel electrophoresis (2D-DIGE) can be used, in which different protein samples are labelled with fluorescent dyes prior to 2D electrophoresis. After the electrophoresis has taken place, the gel is scanned with the excitation wavelength of each dye one after the other. This technique is particularly useful in detecting changes in protein abundance, for example when comparing a sample from a healthy subject and a sample form a diseased subject.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Protein microarrays can also be used to directly detect protein expression. These are similar to DNA and mRNA microarrays in that they comprise capture molecules fixed to a solid surface. Capture molecules are most commonly antibodies specific to the proteins being detected, although antigens can be used where antibodies are being detected in serum. Further capture molecules include proteins, aptamers, nucleic acids, receptors and enzymes, which might be preferable if commercial antibodies are not available for the protein being detected. Capture molecules for use on the protein arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. There is therefore provided a protein microarray comprising capture molecules (such as antibodies) specific for each of the biomarkers being quantified immobilised on a solid support. In one embodiment of the invention, the microarray comprises capture molecules specific for each of LYVE1, REG1 (REG1A and/or REG1B) and TFF1 proteins.

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltametry and amperometry methods) and radio frequency methods (for example multipolar resonance spectroscopy).

Additional methods of determine protein concentration include mass spectrometry and/or liquid chromatography, such as LC-MS, UPLC, or a tandem UPLC-MS/MS system.

Once the level of expression or concentration has been determined, the level can be compared to a threshold level or previously measured level of expression or concentration (either in a sample from the same subject but obtained at a different point in time, or in a sample from a different subject, for example a healthy subject, i.e. a control or reference sample) to determine whether the level of expression or concentration is higher or lower in the sample being analysed. Hence, the methods of the invention may further comprise a step of correlating said detection or quantification with a control or reference to determine if PDAC is present (or suspected) or not. Said correlation step may also detect the presence of particular types of PDAC and to distinguish these patients from healthy patients, in which no PDAC or pancreatic cancer is present, or from patients suffering from CP or intraductal papillary mucinous neoplasms (IPMNs) or other cystic lesions. For example, the methods may detect early stage PDAC, in particular stage I and/or stage II PDAC, but most preferably stage I PDAC. Said step of correlation may include comparing the amount (expression or concentration) of one, two, or three or more of the panel biomarkers with the amount of the corresponding biomarker(s) in a reference sample, for example in a biological sample taken from a healthy patient. Generally the methods of the invention do not include the steps of determining the amount of the corresponding biomarker in a reference sample, and instead such values will have been previously determined. However, in some embodiments the methods of the invention may include carrying out the method steps from a healthy patient who is used as a control. Alternatively, the method may use reference data obtained from samples from the same patient at a previous point in time. In this way, the effectiveness of any treatment can be assessed and a prognosis for the patient determined.

Internal controls can be also used, for example quantification of one or more different miRNAs or proteins not part of the biomarker panel. This may provide useful information regarding the relative amounts of the biomarkers in the sample, allowing the results to be adjusted for any variances according to different populations or changes introduced according to the method of sample collection, processing or storage.

As would be apparent to a person of skill in the art, any measurements of analyte concentration or expression may need to be normalised to take in account the type of test sample being used and/or and processing of the test sample that has occurred prior to analysis. Data normalisation also assists in identifying biologically relevant results. Invariant miRNAs may be used to determine appropriate processing of the sample. Differential expression calculations may also be conducted between different samples to determine statistical significance.

In general, the methods of the present invention may comprise the steps of:
a) providing a biological sample, such as a urine sample;
b) optionally processing the sample, for example to enrich the sample for miRNAs;
c) extraction of the miRNA(s) of interest from the sample; and
d) quantification of the miRNAs.

The methods may further comprise the step of:
e) comparison of the level of miRNA expression from step d) with a control or reference sample.

In some embodiments of the invention, the step of quantification may comprise the following steps:
a) contacting the sample or extracted miRNAs with a binding partner that specifically binds to the miRNA(s) of interest
b) quantifying the amount of miRNA-binding partner to determine the amount of the miRNA(s) present in the original sample.

The present invention therefore provides a reaction mixture, comprising either the miRNAs of interest, or a biological sample (such as a urine sample) containing the miRNAs of interest, wherein each of the miRNAs of interest are bound to a binding partner specific to the miRNA. The binding partner may be, for example, an oligonucleotide that hybridises to the miRNA

Alternatively, the reaction mixture may comprise cDNA molecules corresponding to the miRNAs of interest, and it is the cDNAs that are bound to a specific binding partner.

The test may be combined with a protein biomarker panel to increase its reliability. For example, the test may be combined with a biomarker panel comprising a protein selected from the group consisting of LYVE1, REG1 (REG1A and/or REG1B) and TFF1. Alternatively, or in addition, CA19.9 may also be included in the biomarker panel to further reduce the incidence of false positives or false negatives. There is therefore provided a biomarker panel comprising one or more miRNAs selected from the group consisting of miR-143, miR-223, miR-204. miR-30e, miR-26a, miR-30a, miR-30b and miR-106b, one or more proteins selected from the group consisting of LYVE1, REG1 and TFF1, and optionally the protein CA19.9. In a preferred embodiment, there is provided a biomarker panel comprising one or more mRNAs selected from the group consisting of miR-143, miR-223, miR-204, miR-30e, miR-30b and miR106b, one or more proteins selected from the group consisting of LYVE1, REG1 and TFF1, and optionally the protein CA19.9. References herein to REG1 include REG1A and/or REG1B.

In some embodiments of the invention, the method comprises correlating the measured biomarkers with PDAC, in particular stage I PDAC (depending on the biomarkers being used, as discussed above). The present invention therefore provides a method of qualifying pancreatic disease in a patient, or determining the presence or absence of pancreatic disease, the method comprising measuring the abundance of one or more relevant biomarkers in a biological sample (such as a urine sample) and correlating the measured biomarkers with a stage of disease. The stage of disease may be chronic pancreatitis, stage I PDAC, or a later stage of PDAC. Alternatively, it may be determined the patient is healthy, i.e. pancreatic disease is absent.

The method of the invention can be carried out using a binding molecules or reagents specific for the miRNAs, proteins or cDNAs being detected. Binding molecules and reagents are those molecules that have an affinity for the target such that they can form binding molecule/reagent-biomarker complexes that can be detected using any method known in the art. The binding molecule of the invention can be an antibody, an antibody fragment, a nucleic acid, an oligonucleotide, a protein or an aptamer or molecularly imprinted polymeric structure, depending on the nature of the target (for example miRNA or, in some embodiments, cDNA or protein). Methods of the invention may comprise contacting the biological sample with an appropriate binding molecule or molecules. Said binding molecules may form part of a kit of the invention, in particular they may form part of the biosensors of in the present invention.

Antibodies can include both monoclonal and polyclonal antibodies and can be produced by any means known in the art. Techniques for producing monoclonal and polyclonal antibodies which bind to a particular protein are now well developed in the art. They are discussed in standard immunology textbooks, for example in Roitt et al., Immunology, second edition (1989), Churchill Livingstone, London. Polyclonal antibodies can be raised by stimulating their production in a suitable animal host (e.g. a mouse, rat, guinea pig, rabbit, sheep, chicken, goat or monkey) when the antigen is injected into the animal. If necessary, an adjuvant may be administered together with the antigen. The antibodies can then be purified by virtue of their binding to antigen or as described further below. Monoclonal antibodies can be produced from hybridomas. These can be formed by fusing myeloma cells and B-lymphocyte cells which produce the desired antibody in order to form an immortal cell line. This is the well known Kohler & Milstein technique (Kohler & Milstein (1975) Nature, 256:52-55). The antibodies may be human or humanised, or may be from other species.

The present invention includes antibody derivatives which are capable of binding to antigen. Thus the present invention includes antibody fragments and synthetic constructs. Examples of antibody fragments and synthetic constructs are given in Dougall et al. (1994) Trends Biotechnol, 12:372-379.

Antibody fragments or derivatives, such as Fab, F(ab′)₂ or Fv may be used, as may single-chain antibodies (scAb) such as described by Huston et al. (993) Int Rev immunol, 10:195-217, domain antibodies (dAbs), for example a single domain antibody, or antibody-like single domain antigen-binding receptors. In addition antibody fragments and immunoglobulin-like molecules, peptidomimetics or non-peptide mimetics can be designed to mimic the binding activity of antibodies. Fv fragments can be modified to produce a synthetic construct known as a single chain Fv (scFv) molecule. This includes a peptide linker covalently joining VH and VL regions which contribute to the stability of the molecule. The present invention therefore also extends to single chain antibodies or scAbs.

Other synthetic constructs include CDR peptides. These are synthetic peptides comprising antigen binding determinants. These molecules are usually conformationally restricted organic rings which mimic the structure of a CDR loop and which include antigen-interactive side chains. Synthetic constructs also include chimeric molecules. Thus, for example, humanised (or primatised) antibodies or derivatives thereof are within the scope of the present invention. An example of a humanised antibody is an antibody having human framework regions, but rodent hypervariable regions. Synthetic constructs also include molecules comprising a covalently linked moiety which provides the molecule with some desirable property in addition to antigen binding. For example the moiety may be a label (e.g. a detectable label, such as a fluorescent or radioactive label) or a pharmaceutically active agent.

In those embodiments of the invention in which the binding molecule is an antibody or antibody fragment, the method of the invention can be performed using any immunological technique known in the art. For example, ELISA, radio immunoassays, bead-based, or similar techniques may be utilised. In general, an appropriate autoantibody is immobilised on a solid surface and the sample to be tested is brought into contact with the autoantibody. If the cancer biomarker recognised by the autoantibody is present in the sample, an antibody-marker complex is formed. The complex can then be directed or quantitatively measured using, for example, a labelled secondary antibody which specifically recognises an epitope of the biomarker. The secondary antibody may be labelled with biochemical markers such as, for example, horseradish peroxidase (HRP) or alkaline phosphatase (AP), and detection of the complex can be achieved by the addition of a substrate for the enzyme which generates a colorimetric, chemiluminescent or fluorescent product. Alternatively, the presence of the complex may be determined by addition of a protein labelled with a detectable label, for example an appropriate enzyme. In this case, the amount of enzymatic activity measured is inversely proportional to the quantity of complex formed and a negative control is needed as a reference to determining the presence of antigen in the sample. Another method for detecting the complex may utilise antibodies or antigens that have been labelled with radioisotopes followed by a measure of radioactivity. Examples of radioactive labels for antigens include ³H, ¹⁴C and ¹²⁵I.

Aptamers are oligonucleotides or peptide molecules that bind a specific target molecule. Oligonucleotide aptamers include DNA aptamers and RNA aptamers. Aptamers can be created by an in vitro selection process from pools of random sequence oligonucleotides or peptides. Aptamers can be optionally combined with ribozymes to self-cleave in the presence of their target molecule.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target biomarker before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target biomarker is monitored cycle by cycle. An enriched pool is cloned once it is judged that the proportion of binding molecules has risen to an adequate level. The binding molecules are then analysed individually. SELEX is reviewed in Fitzwater & Polisky (1996) Methods Enzymol, 267:275-301.

Thus, in one embodiment of the invention, there is provided a method of analysing a biological sample (such as a urine sample) from a patient, comprising contacting the sample with reagents or binding molecules specific for the biomarker(s) being quantified, and measuring the abundance of biomarker-reagent or biomarker-binding molecule complexes, and correlating the abundance of biomarker-reagent or biomarker-binding molecule complexes with the concentration of the relevant biomarker in the biological sample. For example, in one embodiments of the invention, the method comprises the steps of:
a) contacting a biological sample (such as a urine sample) with reagents or binding molecules specific for one or more of miR-143, miR-223, miR-204, miR-30e, miR-26a, miR-30a, miR-30b and miR-106b (such as one or more of miR-143, miR-223, miR-204 and miR-30e);
b) quantifying the abundance of biomarker-reagent or biomarker-binding molecule complexes for the one or more miRNAs; and
c) correlating the abundance of biomarker-reagent or biomarker-binding molecule complexes with the concentration of the one or more miRNAs in the biological sample.

The method may further comprise the step of d) comparing the concentration of the biomarkers in step c) with a reference to determine the presence or absence of PDAC. The patient can then be treated accordingly. Depending on the method being undertaking, the biomarkers may determine the presence of absence of CP. The patient can then be treated accordingly.

In some embodiments of the invention, the methods comprise contacting the biological sample with reagents or binding molecules specific for one, two, three or four or more of miR-143, miR-223, miR-204, miR-30e, miR-26a, miR-30a, miR30b and miR-106b. In some embodiments of the invention, the methods comprise contacting the biological sample with reagents or binding molecules specific for one, two, three or four of miR-143, miR-223, miR-204 and miR-30e. In further embodiments, as noted above, the miRNA biomarkers may be combined with one or more of LYVE1, REG1 (REG1A and/or REG1B) and TFF1, and optionally CA19.9 may be included, by additionally contacting the biological sample with a reagent or binding molecule specific for the biomarker protein(s). Suitable reagents or binding molecules may include an antibody or antibody fragment, an enzyme, a nucleic acid, an organelle, a cell, a biological tissue, imprinted molecule or a small molecule. Such methods may be carried out using kits or biosensors of the invention.

The present invention also provides a method of diagnosis for pancreatic ductal adenocarcinoma comprising detecting the level of expression or concentration of a biomarker selected from the group consisting of miR-143, miR-223, miR-204, miR-30e, miR30b and miR-106b, or combinations thereof, in a biological sample, in particular a urine sample. In one embodiment of the invention, the method may comprise detecting the level of expression of at least two, at least three or at least four biomarkers selected from the group consisting of miR-143, miR-223, miR-204, miR-30e, miR30b and miR106b, in a biological sample, in particular a urine sample. In one embodiment of the invention, the method may comprise detecting the level of expression of at least two, at least three or at least 4 biomarkers selected from the group consisting of miR-143, miR-223, miR-204 and miR-30e, in a biological sample, in particular a urine sample. Since some of the biomarker panels of the invention can also detect chronic pancreatitis, analogous methods of diagnosis of CP are also provided using a biomarker or biomarker panel that is useful in detecting CP. For example, in one embodiment of the invention relating to diagnosis of CP, the method may comprise detecting the level of expression of at least two, at least three or at least four biomarkers selected from the group consisting of miR-223, miR-204, miR-26a, miR-30a, miR-30b and miR106b.

The presence of pancreatic ductal adenocarcinoma can be determined by detecting an increase in biomarker expression or protein concentration as compared with the level of expression or protein concentration of the corresponding biomarkers in samples taken from healthy control subjects. In addition, the level of expression or concentration can be used to distinguish between PDAC and CP. This can be achieved by comparing the level of expression or concentration found in the test sample with that seen in patients presenting with CP (or to a reference). Furthermore, the biomarkers can be used to distinguish between PDAC and intraductal papillary mucinous tumours (IPMNs) or any other cystic lesion. This can be done by comparing the level of expression or concentration found in the test sample with that seen in patients presenting with IPMNs (or to a reference).

In a further embodiment of the invention there is provided a miRNA selected from the group consisting of miR-143, miR-223, miR-204 and miR-30e, or a combination therefor, for use in diagnosing PDAC. In some embodiments, these miRNA biomarkers may be combined with one or more protein biomarkers selected from the group consisting of LYVE1, REG1 (REG1A and/or REG1B) and TFF1, or a combination thereof. In one embodiment of the invention, there is provided the combination of two of miR-143, miR-223, miR-204 and miR-30e for use in the diagnosis of PDAC. There is also provided the combination of all three of miR-143, miR-223 and miR-30e or the combination of all three of miR-143, miR-223 and miR-204 for use in the diagnosis of PDAC. These biomarker panels may additionally be combined with the protein biomarker CA19.9 in some embodiments of the present invention, or the other protein biomarkers discussed herein.

In another embodiment of the invention there is provided a method of treating or preventing PDAC in a patient, comprising quantifying one or more biomarkers selected from the group consisting of miR-143, miR-223, miR-204 and miR-30e in a biological sample (in particular a urine sample) obtained from a patient, comparing the values to a reference for each of the quantified biomarkers, and, if the detected values are greater than the reference, administering treatment for PDAC. Methods of treating PDAC may include resecting the pancreatic tumour and/or administering chemotherapy and/or radiotherapy to the patient. In some embodiments, each of miR-143, miR-223 and miR-30e, or each of miR-143, miR-223 and miR-204, or each of miR-223 and miR-204 are quantified. Quantification may also comprise quantifying one or more of the protein biomarkers LYVE1, REG1 (REG1A and/or REG1B) and TFF1. CA19.9 may also be quantified in such methods. Methods of treatment may be carried out on patients that have been diagnosed or are suspected as having PDAC based on the results of the diagnostic test described herein.

In a still further embodiment of the invention there is provided a method for determining the suitability of a patient for treatment for PDAC or CP, comprising detecting the level of expression of an miRNA selected from the group consisting of miR-143, miR-223, miR-204, miR-30e, miR30b and miR106b (optionally the group consisting of miR-143, miR-223, miR-204 and miR-30e), or combinations thereof, in a urine sample, comparing the level of expression with a control, and deciding whether or not to proceed with treatment for PDAC or CP if PDAC or CP is diagnosed or suspected. The method may also comprise detecting the level of expression of a protein biomarker selected from the group consisting of LYVE1, REG1 (REG1A and/or REG1B) and TFF1, plus comparison with relevant controls. The method may further comprise detecting the level of expression of CA19.9, and comparison with a relevant control.

In some embodiments of the invention, the methods may further comprise treating a patient for PDAC or CP if PDAC or CP is detected or suspected. If PDAC or CP is detected or suspect based on the analysis of a urine, blood or serum sample, the presence of PDAC or CP can be confirmed by, for example, detecting the presence and/or amount of the biomarkers in a sample of pancreatic tissue. Methods of the invention may therefore further comprise a step of detecting or determining the amount of a biomarker in a pancreatic tissue sample. The pancreatic tissue sample may have been obtained previously from a patient, or the method may comprise a step of obtaining or providing said pancreatic tissue sample. Analysis of pancreatic tissue samples may also comprise a histological analysis.

If possible, treatment for PDAC (in particular stage I and stage II PDAC) involves resecting the tumour. Treatment may alternatively or additional involve treatment by chemotherapy and/or radiotherapy. Treatment by chemotherapy may include administration of gemcitabine and/or Folfirinox. Folfirinox is a combination of fluorouracil (5-FU), irinotecan, oxaliplatin and folinic acid (leucovorin). Treatment regimens involving Folfirinox may comprise administration of oxaliplatin, followed by folinic acid, followed by irinotecan (alternatively irinotecan may be administered at the same time as folinic acid), followed by 5-FU.

In some embodiments, the methods of treatment are performed on patients who have been identified as having a particular concentration of the biomarker in a biological sample. Said concentration is one that it is indicative of PDAC, such as early stage PDAC. Methods of treating CP are also provided.

There is also provided a method of monitoring a patient's response to therapy, comprising determining the level of expression of at least one of the biomarkers of interest in a biological sample obtained from a patient that has previously received therapy PDAC (for example chemotherapy and/or radiotherapy). In some embodiments, the level of expression is compared with the level of expression for the same biomarker or biomarkers in a sample obtained from a patient before receiving the therapy. A decision can then be made on whether to continue the therapy or to try an alternative therapy based on the comparison of the levels of expression.

In one embodiment, there is therefore provided a method comprising:
a) determining the level of expression of at least one biomarker or combination of biomarkers of the invention in a biological sample obtained from a patient that has previously received therapy for pancreatic cancer or PDAC;
b) comparing the level of expression of the biomarker or biomarkers determining in step a) with a previously determined level of expression of the same biomarker or biomarkers; and
c) maintaining, changing or withdrawing the therapy for pancreatic cancer or PDAC.

The method may comprise a prior step of administering the therapy for pancreatic cancer or PDAC to the patient. In another embodiment, the method may also comprise a pre-step of determining the level of expression of at least one biomarker or combination of biomarkers of the invention in a biological sample obtained from the same patient prior to administration of the therapy. In step c), the therapy for pancreatic cancer or PDAC may be maintained if an appropriate adjustment in the level(s) of expression of the biomarker or biomarkers is determined. For example, if there is a reduction in the expression of one or more of the biomarkers found to be up-regulated in pancreatic cancer or PDAC, then treatment may be maintained. If the levels of expression have altered sufficiently, for example back to what may be considered healthy or low-risk levels, then treatment for pancreatic cancer or PDAC may be withdrawn. If the levels of expression are unchanged or have worsened (for example there is an increase in the expression of one or more of the biomarkers found to be up-regulated in pancreatic cancer or PDAC), this may be indicative of a worsening of the patient's condition, and hence an alternative therapy for pancreatic cancer or PDAC may be attempted. In this way, drug candidates useful in the treatment of pancreatic cancer or PDAC (in particular early stage PDAC) can be screened.

In another embodiment of the invention, there is provided a method identifying a drug useful for the treatment of PDAC, comprising:
(a) quantifying the expression or concentration of one or more biomarkers selected from the group consisting of miR-143, miR-223, miR-204, miR-30e, miR30b and miR-106b (optionally selected from the group consisting of miR-143, miR-223, miR-204 and miR-30e) in a biological sample obtained from a patient;
(b) administering a candidate drug to the patient;
(c) quantifying the expression or concentration of one or more biomarkers selected from the group consisting of miR-143, miR-223, miR-204, miR-30e, miR30b and miR-106b (optionally selected from the group consisting of miR-143, miR-223, miR-204 and miR-30e) in a biological sample obtained from the same patient at a point in time after administration of the candidate drug; and
(d) comparing the value determined in step (a) with the value determined in step (c), wherein a decrease in the level of expression or concentration of one or more of the biomarkers between the two samples identifies the drug candidate as a possible treatment for PDAC. In some embodiments, the method uses all of the panel biomarkers miRNAs, i.e. all of miR-143, miR-223, miR-204, miR-30e, miR30b and miR-106b (optionally all of miR-143, miR-223, miR-204 and miR-30e) must be quantified in step (a) and step (c). In some embodiments, the biological sample is a urine sample. In some embodiments, the drug is a compound, an antibody or antibody fragment. In some embodiments of the invention, the miRNAs quantified in steps (a) and (b) are the same.

### Kits and Biosensors

In a still further embodiment of the invention there is provided a kit of parts for testing for pancreatic ductal adenocarcinoma comprising a means for quantifying the expression or concentration of (i.e. measuring) miR-143, miR-223, miR-204, miR-30e, miR-26a, miR-30a, miR-30b and miR-106b, or combinations thereof. The means may be any suitable detection means that can measure the quantity of biomarkers in the sample.

In one embodiment, the means may be a biosensor. In some embodiments, the means may comprise a dipstick coated with a membrane to that is bound an unlabelled binding molecule (such as an antibody or antibody fragment) with specific affinity for the biomarker being detected on a first section. The membrane may also have a section that is blocked with a non-reactive molecule to prevent any molecules binding to that part of the membrane. The membrane may also have a section to which is bound the biomarker that is being detected in the sample. The dipstick may be equipped to detect more than one biomarker in a single assay by having different sections dedicated to the detection of different biomarkers of the invention, such that each further biomarker to be detected has a corresponding antibody capable of specifically binding that further biomarker bound on one section of the dipstick, and optionally the further biomarker to be detected bound on another section of the dipstick. The kit may also comprise a container for the sample or samples and/or a solvent for extracting the biomarkers from the biological sample. The kits of the present invention may also comprise instructions for use.

In some embodiments of the invention, there is provided a kit of parts for diagnosing pancreatic ductal adenocarcinoma comprising a means for detecting the expression of at least one or at least two of miR-143, miR-223, miR-204, miR-30e, miR-30b and miR-106b (for example miR-143 and miR-30e or miR-223 and miR-204). In further embodiments of the invention, there is provided a kit of parts for diagnosing pancreatic ductal adenocarcinoma or CP comprising a means for detecting the expression of all three of miR-143, miR-223 and miR-30e. In a still further embodiment of the invention, there is provided a kit of parts for diagnosing pancreatic ductal adenocarcinoma or CP comprising a means for detecting the expression of all three of miR-143, miR-223 and miR-204. In a further embodiment of the invention, there is provided a kit of parts for diagnosing CP, comprising a means for detecting the expression of at least two of miR-26a, miR-30a, miR-30b and miR-106b. The means for detecting the biomarkers may be reagents that specifically bind to or react with the biomarkers being quantified.

The kit of parts of the invention may comprise a biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as an miRNA, a cDNA or a protein).

The bioreceptor specifically interacts with or binds to the analyte of interest and may be, for example, an antibody or antibody fragment, an enzyme, a nucleic acid, an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as DNA), antibody, epigenetic, organelle, cell, tissue or microbial biosensors).

Dipsticks are another example of biosensor. The dipsticks of the invention may comprise a membrane. The dipsticks may further comprise a first section to which is bound an unlabelled binding partner (such as antibody) with specific affinity for the biomarker whose expression is being detected, a second section that is blocked with a non-reactive ligand/antigen and optionally a third section to which is bound the biomarker whose expression is being detected.

Dipstick techniques known in the art can be used to quickly and effectively carry out the method of the invention. Dipstick techniques include the following. A labelled binding molecule, such as antibody, for example labelled with formazan, having a specific affinity for the biomarker (antigen) being detected is dissolved in a sample of test fluid. A dipstick on which a nitrocellulose membrane is mounted is immersed in the reaction mixture. The membrane has one section on which non-labelled antibodies having a specific affinity for that antigen are bound. The second section is free of antibodies and is blocked with a non-reactive molecule to prevent binding of labelled antibodies to the membrane. A third section of the dipstick is provided on which the antigen is bound. Reactions take place between the free antigen in the test fluid and the non-labelled antibody bonded to the membrane, as well as between the free antigen and the labelled antibody that was added to the sample. This results in a sandwich of non-labelled bonded antibody/antigen/labelled antibody over the first section of the membrane. A reaction also takes place between the labelled antibody and the bound antigen over the third section. No reaction takes places over the second section of the membrane.

The reaction is allowed to proceed for a fixed period of time or until completion is determined visually. Since formazan is a highly coloured dye, the reacted formazan-labelled antibody imparts colour to the third section, and if the antigen is present in the test fluid, to the first section as well. Since no reaction takes place over the second section, no colour is developed over that section. The second section thus acts as a negative control. In cases in which colour is imparted across the entire membrane, including the second section due to absorption of un-reacted formazan particles and, to a minor extent, of un-reacted formazan-labelled antibody, presence of the antigen is indicated by a difference in colour between the first and second sections of the membrane. The third section is provided as a positive control by demonstrating that the appropriate reactions are in fact taking place.

The length of time that the dipstick is immersed in the mixture is that which allows a difference in colour intensity to develop between the first and second sections of the membrane if the antigen is present. For most antibody-antigen reactions, colour development is essentially complete within 30 to 60 minutes. If desired, colour development of the dipstick can be monitored by simply removing the dipstick, visually checking the colour intensity across the first section of the membrane, and then re-immersing the dipstick if required. When no further change in colour intensity is seen, the reaction can be deemed complete.

The dipstick can be prepared by any conventional methods known in the art. For example, a nitrocellulose membrane is mounted at the lower end of the dipstick. A solution containing non-labelled primary antibody is applied over one section of the membrane to bind primary antibodies to the membrane. A solution containing a blocking agent (for example 1% serum albumin) is applied over another section of the membrane to prevent subsequent bonding of the primary antigen to the membrane.

Dipsticks can be equipped for the detection of more than one biomarker at a time by including further sections to which are bound un-labelled antibodies with specific affinity for the further biomarker or biomarkers being detected and, optionally, a section to which is bound the biomarker being detected. In such cases, labelled antibodies with specific affinity for the biomarker being detected can be added to the sample such that their binding to the further section of the dipstick, and hence their presence in the sample, be detected. The antibodies can be labelled with the same dye or with a different dye. Suitable dyes, other than formazan, include acid dyes (for example anthraquinone or triphenylmethane), azo dyes (for example methyl orange or disperse orange 1), fluorescent dyes (for example fluorescein or rhodamine) or any other suitable dye known in the art such as coomassie blue, amido black, toluidine blue, fast green, Indian ink, silver nitrate and silver lactate. It is also apparent that the pre-labelled primary biomarker reactant is not limited to antibodies, but can include any molecule having specific affinity for a second biomarker to be detected in a sample.

The invention also provides microarrays (RNA, DNA or protein) comprising capture molecules (such as RNA or DNA oligonucleotides) specific for each of the biomarkers or biomarker panels being quantified, wherein the capture molecules are immobilised on a solid support. The microarrays are useful in the methods of the invention.

In particular, the present invention provides a combination of binding molecules, wherein the each binding molecule specifically binds a different target analyte, and the combination of analytes the binding molecules specifically bind to is:
a) miR-143 and miR-30e;
b) miR-223 and miR204;
c) miR-143, miR-223 and miR-30e;
d) miR-143, miR-223 and miR-204;
e) or any other combination of miRNA biomarkers disclosed herein, as dictated by the context (for example the type of pancreatic disease being diagnosed).

For example, a kit comprising a binding molecule that specifically binds to miR-143 and a binding molecule that specifically binds to miR-30e is provided. A kit comprising a binding molecule that specifically binds to miR-223 and a binding molecule that specifically binds to miR-204 is also provided. A kit comprising a comprising a binding molecule that specifically binds to miR-143, a binding molecule that specifically targets miR-223, and a binding molecule that specifically binds to miR-30e is also provided. A kit comprising a comprising a binding molecule that specifically binds to miR-143, a binding molecule that specifically targets miR-223, and a binding molecule that specifically binds to miR-204 is also provided. A kit comprising binding molecules that specifically bind to each of the miRNAs in any biomarker combination disclosed herein, as dictated by the context (for example the type of pancreatic disease being diagnosed), is also provided.

The binding molecules may be present on a solid substrate, such an array (for example an RNA microarray, in which case the binding molecules are RNAs that hybridise to the target miRNA). The binding molecules may all be present on the same solid substrate. Alternatively, the binding molecules may be present on different substrates. In some embodiments of the invention, the binding molecules are present in solution.

These kits may further comprise additional components, such as a buffer solution. Other components may include a labelling molecule for the detection of the bound miRNA and so the necessary reagents (i.e. enzyme, buffer, etc) to perform the labelling; binding buffer; washing solution to remove all the unbound or non-specifically bound miRNAs. Hybridisation will be dependent on the size of the putative binder, and the method use may be to be determined experimentally, as is standard in the art. As an example, hybridisation can be performed at ˜20° C. below the melting temperature (Tm), over-night. (Hybridisation buffer: 50% deionised formamide, 0.3 M NaCl, 20 mM Tris-HCl, pH 8.0, 5 mM EDTA, 10 mM phosphate buffer, pH 8.0, 10% dextran sulfate, 1×Denhardt's solution, and 0.5 mg/mL yeast tRNA). Washes can be performed at 4-6° C. higher than hybridization temperature with 50% Formamide/2×SSC (20× Standard Saline Citrate (SSC), pH 7.5: 3 M NaCl, 0.3 M sodium citrate, the pH is adjusted to 7.5 with 1 M HCl). A second wash can be performed with 1×PBS/0.1% Tween 20.

Binding or hybridisation of the binding molecules to the target analyte may occur under standard or experimentally determined conditions. The skilled person would appreciate what stringent conditions are required, depending on the biomarkers being measured. The stringent conditions may include a hybridisation buffer that is be high in salt concentration, and a temperature of hybridisation high enough to reduce non-specific binding.

In one embodiment of the invention, the kit is able to simultaneously measure both miRNA biomarkers and protein biomarkers.

In one embodiment of the invention, there is provided a method of diagnosing early-stage pancreatic ductal adenocarcinoma (PDAC) comprising determining the expression levels of each of miR-143, miR-223 and either miR-204 or miR-30e in a urine sample and comparing the so determined values to a reference. If the concentration of each of the miRNAs is greater than a reference value, early-stage PDAC may be present, and the patient can be treated accordingly.

Features for the second and subsequent aspects of the invention are as for the first aspect of the invention, mutatis mutandis.

The present invention shall now be further described with reference to the following examples, which are present for the purposes of illustration only and are not to be construed as being limiting on the invention.

### Examples—Identification and Evaluation of miRNA Biomarkers

### Samples

The study was performed using urine specimens collected at the Barts and the London HPB Centre, The Royal London Hospital, and University College Hospital, London, after obtaining patients' consent and with full Ethical approval (REC reference number 05/Q0408/65). The samples were kept on ice upon collection, aliquoted before freezing within two hours of collection and maintained at −80° C. for long-term storage. Both collection and storage were performed according to standard operating procedures, compliant with Tissue bank requirements under Human Tissue Act.

A total of 101 urine samples were analysed in this study, which included 46 PDAC at different stages of disease, 29 chronic pancreatitis (CP), and 26 healthy individuals (H) (Table 1). In addition to verification of clinical notes for the absence of any renal diseases, all urine samples were also first routinely tested with Urine dipstick (Combur10Test, Roche) in order to eliminate samples with pathological values in any of the parameters measured (protein, glucose, bilirubin, leukocytes, specific gravity).

TABLE 1
Demographics of analysed healthy and patient groups.
a) Microarray analysis (Total n = 26*)

| | | | | PDAC (n = 13) | |
|---|---|---|---|---|---|
| | | | | Stage | |
| | | H | CP | I | II-IV |
| | | (n = 7) | (n = 6) | (n = 4) | (n = 9) |
| Average Age (y) | | 61.6 | 69.5 | 64.6 | |
| | <60 | 3 | 0 | 1 | 3 |
| | 60-70 | 3 | 3 | 1 | 3 |
| | >70 | 1 | 3 | 2 | 3 |
| Gender | Female | 4 | 3 | 3 | 5 |
| | Male | 3 | 3 | 1 | 4 |
| Diabetes | | 0 | 0 | 0 | 0 |

b) RT-PCR validation (Total n = 75)

| | | | | PDAC (n = 33) | |
|---|---|---|---|---|---|
| | | | | Stage | |
| | | H | CP | I | II-IV |
| | | (n = 19) | (n = 23) | (n = 2) | (n = 31) |
| Average Age (y) | | 60.4 | 57.7 | 63.9 | |
| | <60 | 9 | 14 | 0 | 9 |
| | 60-70 | 6 | 5 | 1 | 15 |
| | >70 | 4 | 4 | 1 | 7 |
| Gender | Female | 10 | 7 | 0 | 10 |
| | Male | 9 | 16 | 2 | 21 |
| Diabetes | | 0 | 5 | 1 | 4 |

*These samples were also used for the RT-PCR validation experiment.

### Low Molecular Weight (LM140 RNA Isolation

LMW RNAs (<200 bp) that include miRNAs and small nucleolar (sno)RNAs were extracted from 3-5 ml of cryopreserved urine, using the Urine microRNA Purification Kit from NORGEN, followed by concentration with RNAstable (Biomatrica). Quantification was performed with Quant-iT OliGreen Kit (Invitrogen), and quality assessed on Bioanalyser using the Agilent Small RNA kit.

Global miRNA Expression Profiling

The microarray profiling was performed using the Affymetrix GeneChip microRNA v. 3.0 Arrays, which comprise 5,607 human small RNAs, including 1,733 mature miRNAs, 1,658 precursor miRNAs and 2,216 snoRNAs. 100 ng of LMW RNAs was labeled using FlashTag Biotin HSR RNA Labeling Kit (Affymetrix) according to the manufacturer's protocol, and ELOSA QC Assay performed in order to confirm the successful labeling. Hybridization to the GeneChip microRNA array, staining, washing and scanning was performed according to standard protocols on Affymetrix workstation.

Raw data files were first analysed with the Expression Console Software v 1.2 (Affymetrix); a quality control (QC) report with information concerning the performance of the experiment was obtained for each array. Samples were normalized using the Robust Multi-Array (RMA) algorithm. Pearson correlation coefficient (r) was used to determine the reproducibility of labeling and hybridization.

### Validation of MicroRNA Expression

Differential miRNA expression was validated by real-time PCR using the TaqMan MicroRNA Assays (Life Technologies) and carried out on a Fluidigm BioMark HD System, a microfluidic platform for high-throughput real-time PCR quantification (Moltzahn F, Hunkapiller N, Mir A A, Imbar T and Blelloch R. High throughput microRNA profiling: optimized multiplex qRT-PCR at nanoliter scale on the fluidigm Dynamic Array™ IFCs. J Vis Exp 2011 and Seumois G, Vijayanand P, Eisley C J, Omran N, Kalinke L, North M, Ganesan A P, Simpson L J, Hunkapiller N, Moltzahn F, Woodruff P G, Fahy J V, Erle D J, Djukanovic R, Blelloch R and Ansel K M, An integrated nano-scale approach to profile miRNAs in limited clinical samples. Am J Clin Exp Immunol 2012; 1:70-89). Two 24.192 Dynamic Array™ Integrated Fluidic Circuits (IFCs) were employed and reactions performed in triplicate. PCR assays were performed as previously described (Jang J S, Simon V A, Feddersen R M, Rakhshan F, Schultz D A, Zschunke M A, Lingle W L, Kolbert C P and Jen J. Quantitative miRNA expression analysis using fluidigm microfluidics dynamic arrays. BMC Genomics 2011; 12: 144). The specific miRNA primers for reverse transcription and pre-amplification reactions were pooled following Life Technologies's instructions (User Bulletin PN 4465407). Multiplexed retro-transcriptions (RT) were carried out using the TaqMan®MicroRNA Reverse Transcription Kit from 20 ng of LMW RNA in 15μl of final volume. To test the limit of sensitivity and the dynamic range of the method, the reverse transcription reaction was performed on a control sample from a healthy individual using an increasing amount of input LMW RNA: 10, 15, 20, 30, 40, 50, 100, and 200 ng.

The pre-amplification reactions were performed from 2.5 μl of RT products in 10μl volume. The mix was first incubated at 95° C. for 10 min, 55° C. for 1 min, followed by 12 cycles of amplification at 95° C. for 15 sec and 60° C. for 4 min. Pre-amplification products were diluted 1:5 in TE 1× and 1.35 μl used to prepare the quantitative PCR reaction mix according to Fluidigm's protocol (192.24_GE_TaqMan_Std PN 100-6170 B1). All the reactions were performed in triplicates.

The sample and the assay mixes were loaded onto a 24.192 Dynamic Array™ Integrated Fluidic Circuits (IFCs), and then placed in the BioMark Instrument for PCR amplification. The chip was first incubated at 95° C. for 10 min, followed by 40 cycles of amplification at 95° C. for 15 sec and 60° C. for 1 min. Data were analysed using the Real-Time PCR Analysis Software, which is integrated in the Fluidigm system. Cₜ values that did not pass the quality threshold of 0.6 (default setting) were discarded.

The average Ct value was calculated for each miRNA assay in each sample. The two plates were scaled and normalized to a value centered around 0 as follows:

[Sample value−mean(plate)]/Standard deviation(plate)

### Statistical Analysis

Data files generated by Affymetrix microarrays were imported into Partek® Genomics Suite™ 6.6 for statistical analysis and hierarchical clustering. Statistically significant differences in miRNAs expression among the examined groups were identified using ANOVA and a 5% false discovery rate (FDR) threshold (Benjamini & Hochberg method (Benjamini Y and Hochberg Y. Controlling the False Discovery Rate: A Practical and Powerful Approach to Multiple Testing. Journal of the Royal Statistical Society. Series B (Methodological) 1995; 57: 289-300)). Hierarchical clustering of the most differentially expressed miRNAs was conducted using Partek default settings.

Normalized Ct values were imported in GraphPad PRISM Software for statistical analysis. The nonparametric Mann-Whitney-U test was applied to calculate the p-values when comparing the miRNA expression levels between two groups.

Selected miRNA biomarkers were investigated for their ability to discriminate between samples from PDAC Stage I patients and healthy control samples using an exploratory Receiver Operating Characteristics (ROC) curve analysis approach based on all available samples. Logistic regression was applied to each miRNA log base 2-transformed average Ct data values. The model was adjusted for plate (experimental run) and the individual's age. ROC curves were generated for each of the miRNAs; the area under the curve (AUC), the sensitivity (SN) and the specificity (SP) at the ‘optimal’ cut-point for discrimination between the two groups were obtained. The optimal cut-point corresponded to the point closest to the top-left part of the plot in the ROC plane (coordinates 0,1) with optimal SN and SP according to the following criterion:

min((1−sensitivities)²+(1−specificities)²),

as calculated by the ‘ci.threshold’ procedure of the R ‘pROC’ package (Robin X, Turck N, Hainard A, Tiberti N, Lisacek F, Sanchez J C and Muller M. pROC: an open-source package for R and S+ to analyze and compare ROC curves. BMC Bioinformatics 2011; 12: 77). This approach has been shown to have good performance in the estimation of the optimal cut-point of a biomarker (Rota M and Antolini L. Finding the optimal cut-point for Gaussian and Gamma distributed biomarkers. Computational Statistics & Data Analysis 2014; 69: 1-14).

MiRNAs were then combined to assess the discriminative power of the combination. MiRNAs that correlated with each other (significant Spearman's correlation coefficient) were not combined to avoid collinearity issues in the model.

Confidence intervals (CI, 95%) for AUCs were derived based on DeLong′ asymptotically exact method to evaluate the uncertainty of an AUC (DeLong E R, DeLong D M and Clarke-Pearson D L. Comparing the areas under two or more correlated receiver operating characteristic curves: a nonparametric approach. Biometrics 1988; 44: 837-845). SN and SP, 95% CI were derived using non-parametric stratified resampling with the percentile method (2,000 bootstrap replicates) as described by Carpenter et al. (Carpenter J and Bithell J. Bootstrap confidence intervals: when, which, what? A practical guide for medical statisticians. Stat Med 2000; 19: 1141-1164). AUCs were compared using DeLong's 1-sided test for correlated/paired (DeLong et al).

ROC curve analyses were performed in R version 2.13.0 (The R Foundation for Statistical Computing, http://www.r-project.org/foundation) using procedures from the Epi (Carstensen B, Plummer M, Laara E and Hills M. Epi: A Package for Statistical Analysis in Epidemiology. R package version 1.1.67. 2014), pROC (Robin et al) Error! Hyperlink reference not valid. and ROCR (Sing T, Sander O, Beerenwinkel N and Lengauer T. ROCR: visualizing classifier performance in R. Bioinformatics 2005; 21: 3940-3941) packages.

### Results

Urine miRNA Expression Profiling

Global LMW RNA expression profile was determined for 26 urine samples, which included 13 PDAC samples (four Stage I, three Stage II, six Stage III-IV), six CP and seven healthy individuals (Table 1a). For one of the healthy samples, two biological replicates (independent RNA extractions) and two technical replicates were also performed to assess the reproducibility of the isolation method and the robustness of the profiling platform, respectively. Therefore, in total, 30 arrays were interrogated. (Microarray data are deposited in GEO, accession number GSE71962). Both the isolation method and the Affymetrix platform proved highly reproducible, resulting in correlation coefficients >0.95 between duplicates (data not shown). The Expression Console Software v 1.2 (Affymetrix) applied to the whole set of experiments, confirmed the expression of an average of 815 (ranging from 748 to 1003) miRNAs per sample. One sample (PDAC Stage III) failed QC and was thus not further analysed.

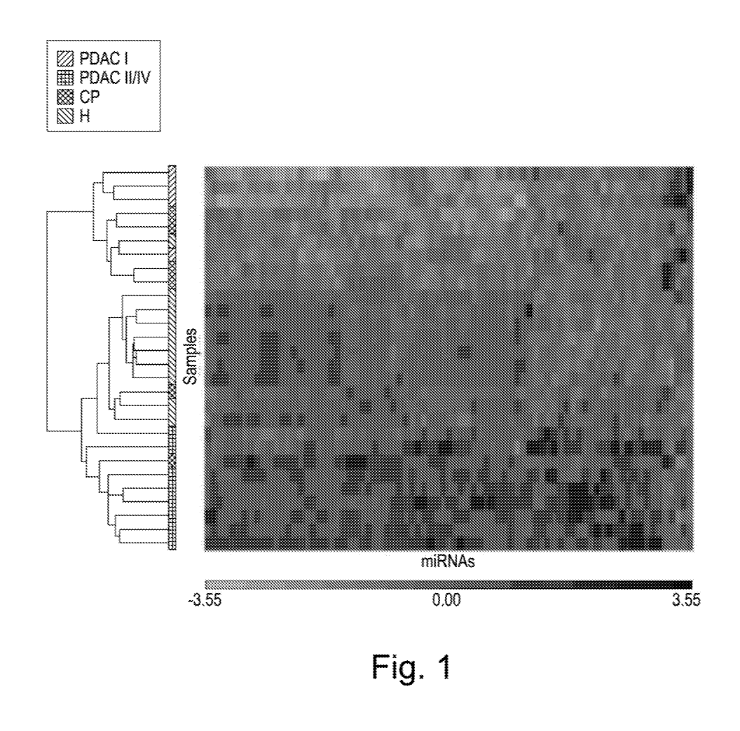

ANOVA applied to identify differentially expressed miRNAs between the sample groups (H, CP and PDAC Stage I and Stage II-IV), led to the identification of 79 statistically significant differentially expressed miRNAs (FDR<0.05). A supervised hierarchical cluster analysis of these 79 miRNA profiles showed clear grouping of the healthy, PDAC stage I and PDAC stage II-IV samples according to their disease status (FIG. 1); in contrast, CP samples showed high heterogeneity and did not cluster.

### Biomarker Selection and Validation by RT-PCR

Out of the 79 differentially expressed miRNAs, urine expression levels of 15 miRNAs showing the lowest p-values (Table 2) were selected for validation.

The 26 samples previously profiled by microarrays, and a further 75 new urine specimens, including 33 PDAC (two Stage I, 31 Stage II-IV), 23 CP and 19 healthy controls (Table 1 b), were interrogated using the TaqMan/Fluidigm BioMark platform. However, three samples (one PDAC Stage II, one PDAC Stage III, and one CP) were excluded from the analysis as the Ct values for the miRNA assays measured failed the quality control.

Among the selected 15 miRNAs, four miRNAs (miR-30e, miR-143, miR-204 and miR-223) were found in significantly higher amounts in the urine of PDAC Stage I patients compared to the healthy population.

These miRNAs (except for miR-204) also showed a decreased expression in Stage II-IV compared to Stage I (Table 2). Another three miRNAs, (miR-3141, miR-4739 and miR-4750) were significantly down regulated in CP compared to healthy, whereas miR-30b was the only miRNA with increased expression in CP compared with both the healthy and PDAC (all stages) groups. The expression of miR-3663-3p, miR-665 and miR-483-5p was significantly higher in later PDAC stages than in healthy samples. The remaining four candidate miRNAs were differentially expressed between PDAC Stages II-IV and I and/or CP (Table 2).

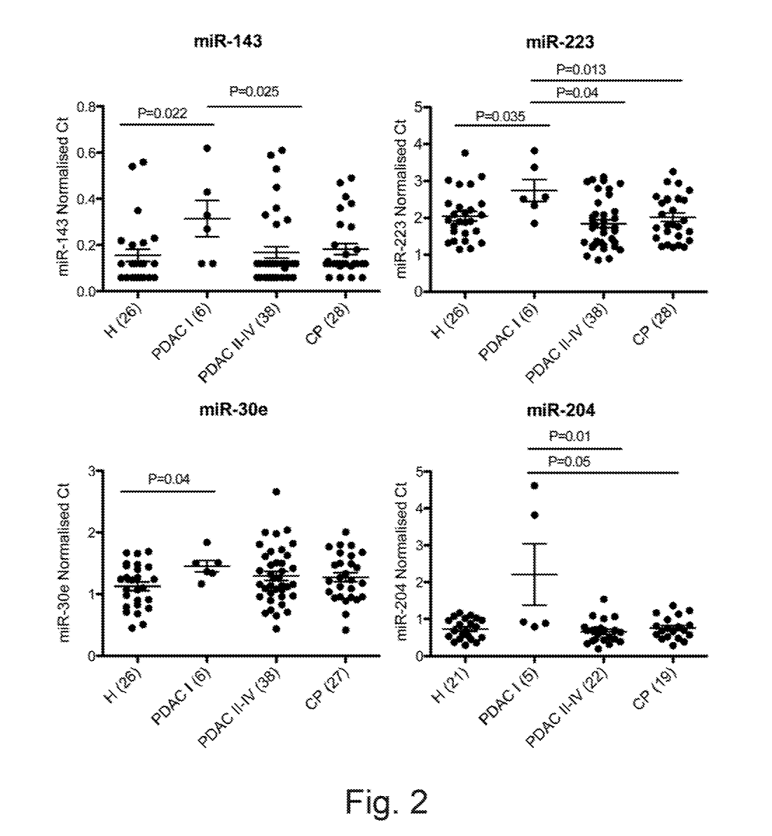

Significant over-expression in PDAC Stage I when compared with healthy controls was confirmed for miR-30e, miR-143 and miR-223. MiR-143, miR-223 and miR-204 also showed statistically higher levels in Stage I when compared to Stages II-IV. Furthermore, miR-223 and miR-204 were also able to distinguish patients with Stage I from patients with CP (FIG. 2). A significant differential expression between PDAC Stage II-IV and CP was also confirmed for miR-1915 (data not shown). The expression of six miRNAs (miR-30b, miR-1207-5p, miR-1275, miR-483-5p, miR-3141, and miR-4739), while correlating with the results obtained by Affymetrix array, resulted in p-values that were just below the threshold for statistical significance. The differential expression of one miRNA, miR-3663-5p, was not confirmed and the remaining three (miR-4750, mir-149*, mir-665) failed the Ct quality filter set by the analysis program.

TABLE 2
List of 15 miRNAs selected for validation. The fold change [FC] and the significance
level of adjusted p-values (P) are reported for every pairwise comparison.

| | St. I v H | | St. II-IV v H | | St. I v II-IV | |
|---|---|---|---|---|---|---|
| | [FC] | (Adj P) | [FC] | (Adj P) | [FC] | (Adj P) |
| miR-30e | Up [2.2] | 0.00002 | Up [1.2] | 0.21 | Up [1.9] | 0.0003 |
| miR-143 | Up [2.0] | 0.0001 | Up [1.1] | 0.62 | Up [1.9] | 0.0004 |
| miR-223 | Up [4.3] | 0.0002 | Up [1.0] | 0.89 | Up [4.2] | 0.0005 |
| miR-204 | Up [1.4] | 0.0004 | Up [1.2] | 0.013 | Up [1.2] | 0.06 |
| miR-30b | Up [1.6] | 0.13 | Up [1.1] | 0.63 | Up [1.4] | 0.27 |
| miR-149* | Down [5.9] | 1.80E−07 | Down [1.3] | 0.15 | Down [4.4] | 3.20E−06 |
| miR-1915 | Down [2.9] | 0.00001 | Up [1.5] | 0.017 | Down [4.2] | 1.80E−07 |
| miR-3141 | Down [7.1] | 1.90E−07 | Down [1.1] | 0.64 | Down [6.5] | 5.60E−07 |
| miR-4739 | Down [3.9] | 0.00002 | Up [3.8] | 0.009 | Down [6.9] | 1.50E−07 |
| miR-4750 | Down [6.4] | 0.00002 | Down [2.1] | 0.012 | Down [3.0] | 0.004 |
| miR-3663-3p | Up [1.3] | 0.5 | Up [17.7] | 3.00E−08 | Down [13.3] | 4.80E−06 |
| miR-665 | Up [1.1] | 0.66 | Up [5.6] | 3.00E−07 | Down [5.0] | 0.00002 |
| miR-483-5p | Down [1.1] | 0.7 | Up [2.0] | 0.00001 | Down [2.1] | 0.00008 |
| miR-1275 | Down [2.0] | 0.016 | Up [3.1] | 0.00004 | Down [6.2] | 1.20E−06 |
| miR-1207-5p | Down [2.0] | 0.12 | Up [2.0] | 0.06 | Down [3.9] | 0.005 |

| | | |
|---|---|---|
| CP v H | CP v St. I | CP v St. II-IV |

| | [FC] | (Adj P) | [FC] | (Adj P) | [FC] | (Adj P) |
|---|---|---|---|---|---|---|
| miR-30e | Up [1.2] | 0.26 | Down [1.9] | 0.002 | Up [1.0] | 0.9 |
| miR-143 | Up [1.2] | 0.14 | Down [1.6] | 0.006 | Up [1.2] | 0.31 |
| miR-223 | Up [1.2] | 0.63 | Down [3.7] | 0.002 | Up [1.1] | 0.73 |
| miR-204 | Down [1.1] | 0.35 | Down [1.5] | 0.0001 | Down [1.3] | 0.003 |
| miR-30b | Up [3.3] | 0.00008 | Up [2.1] | 0.03 | Up [3.0] | 0.0004 |
| miR-149* | Down [2.5] | 0.0008 | Up [2.4] | 0.005 | Down [1.9] | 0.02 |
| miR-1915 | Down [1.4] | 0.07 | Up [2.0] | 0.005 | Down [2.1] | 0.0007 |
| miR-3141 | Down [5.8] | 9.70E−07 | Up [1.2] | 0.51 | Down [5.3] | 2.90E−06 |
| miR-4739 | Down [4.2] | 8.80E−06 | Down [1.1] | 0.8 | Down [7.5] | 8.00E−08 |
| miR-4750 | Down [6.9] | 0.00001 | Down [1.1] | 0.86 | Down [3.2] | 0.003 |
| miR-3663-3p | Down [1.1] | 0.82 | Down [1.5] | 0.45 | Down [19.5] | 6.80E−07 |
| miR-665 | Down [1.3] | 0.4 | Down [1.5] | 0.28 | Down [7.2] | 1.30E−06 |
| miR-483-5p | Down [1.1] | 0.7 | Down [1.0] | 0.99 | Down [2.1] | 0.00008 |
| miR-1275 | Down [3.1] | 0.0004 | Down [1.5] | 0.19 | Down [9.5] | 5.00E−08 |
| miR-1207-5p | Down [4.1] | 0.003 | Down [2.1] | 0.16 | Down [8.1] | 0.0001 |

Diagnostic Potential of the miRNAs to Discriminate Between Healthy and PDAC Stage I Individuals

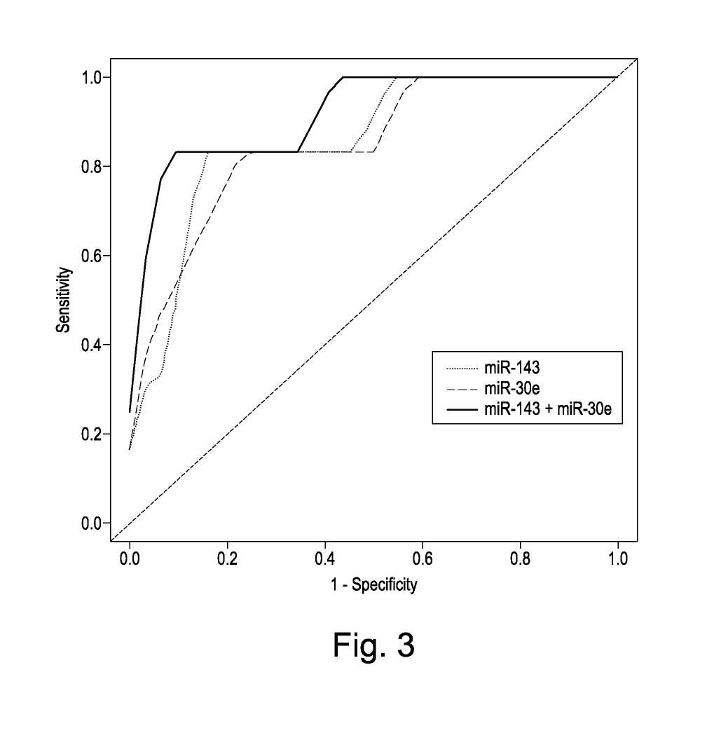

Logistic regression analysis was applied to the Fluidigm data obtained for miR-143, miR-30e and miR-223 (miR-204 was not included in this analysis as RT-PCR did not perform well for several samples). Among the three biomarkers, miR-143 was best able to differentiate Stage I (n=6) from healthy (n=26) (AUC=0.862 (95% CI 0.695-1.000), with SN of 83.3% (95% CI 50.0-100.0) and SP of 88.5% (95% CI 73.1-100.0) at optimal cut-point; Table 3 and FIG. 3). The combination of miR-143 with miR-30e was significantly better at discriminating between the two groups, achieving an AUC of 0.923 (95% CI 0.793-1.000), with SN of 83.3% (95% CI 50.0-100.0) and SP of 96.2% (95% CI 88.5-100.0) at optimal cut-point (Table 3 and FIG. 3). Combining miR-30e with miR-223 only achieved an AUC of 0.891 (95% CI 0.714-1.000), which was not significantly better at the 5% level compared to miR-30e alone (AUC=0.853 (95% CI 0.673-1.000), p=0.1; Table 3 and FIG. 3) although a larger sample size may reveal this increase in AUC to be significant.

TABLE 3
Results of the ROC analyses for the discrimination
between healthy and PDAC stage I individuals.

| | | | |
|---|---|---|---|
| miRNAs | AUC (95% Cl) | % SN (95% Cl)* | % SP (95% Cl)* |

Individual markers

| | | | |
|---|---|---|---|
| miR-143 | 0.862 | 83.3 | 88.5 |
| | (0.695-1.000) | (50.0-100.0) | (73.1-100.0) |
| miR-30e | 0.853 | 83.3 | 80.8 |
| | (0.673-1.000) | (50.0-100.0) | (65.4-96.2) |
| miR-223 | 0.795 | 83.3 | 76.9 |
| | (0.586-1.000) | (50.0-100.0) | (61.5-92.3) |

Combinations^{$}

| | | | |
|---|---|---|---|
| miR-143 + | 0.923 | 83.3 | 96.2 |
| miR-30e^{$$} | (0.793-1.000) | (50.0-100.0) | (88.5-100.0) |
| miR-30e + | 0.891 | 83.3 | 92.3 |
| miR-223^{$$$} | (0.714-1.000) | (50.0-100.0) | (70.8-100.0) |

*at optimal cut-point
^{$}miR-30e did not significantly correlate with miR-223, while miR-143 correlated significantly with miR-223 (p = 0.59, p < 0.001) and resulted in collinearity.
^{$$}DeLong's 1-sided test for correlated/paired AUCs to assess whether the addition of miR-30e significantly increases the AUC obtained with miR-143 alone (0.923 versus 0.862), p = 0.04.
^{$$$}DeLong's 1-sided test for correlated/paired AUCs to assess whether the addition of miR-223 significantly increases the AUC obtained with miR-30e alone (0.891 versus 0.853), p = 0.1.

In this above studies, the feasibility of a genome-wide expression analysis of miRNAs in the urine of patients with PDAC and CP and comparison of them to healthy controls has been demonstrated for the first time. Moreover, the significant over-expression for a subset of miRNAs in PDAC Stage I versus healthy individuals (miR-143, miR-223, miR-30e) and Stage I versus Stages II-IV PDAC (miR-204, miR-143, miR-223) has been established. Prior to the present disclosure, not only had the expression of miRNAs in urine specimens from PDAC patients not previously been interrogated, more importantly, miRNAs capable of detecting PDAC patients at Stage I have not yet been described.

To summarise, three miRNAs (miR-143, miR-223, and miR-30e) were significantly over-expressed in patients with Stage I cancer when compared with age-matched healthy individuals (P=0.022, 0.035 and 0.04, respectively); miR-143, miR-223 and miR-204 were also shown to be expressed at higher levels in Stage I compared to Stages II-IV PDAC (P=0.025, 0.013 and 0.008, respectively). Furthermore, miR-223 and miR-204 were able to distinguish patients with early stage cancer from patients with CP (P=0.037 and 0.036).

Among the three biomarkers, miR-143 was best able to differentiate Stage I (n=6) from healthy (n=26) with area under the curve (AUC) of 0.862 (95% CI 0.695-1.000), with sensitivity (SN) of 83.3% (95% CI 50.0-100.0), and specificity (SP) of 88.5% (95% CI 73.1-100.0). The combination of miR-143 with miR-30e was significantly better at discriminating between these two groups, achieving an AUC of 0.923 (95% CI 0.793-1.000), with SN of 83.3% (95% CI 50.0-100.0) and SP of 96.2% (95% CI 88.5-100.0).

The present disclosure surprisingly demonstrates that miRNA levels in urine can not only distinguish between healthy and diseased individuals, but importantly, can differentiate early from late stage tumors. Using the same samples and an independent urine sample cohort, successful validation of four differentially expressed miRNAs, showing their potential diagnostic value at an early stage of disease, has been achieved.

### Examples—Identification and Evaluation of Protein Biomarkers

### Clinical Specimens

Healthy, CP and PDAC urine specimens were obtained from the Royal London Hospital (RLH) for the discovery phase (n=18) and from RLH and University College London (later on jointly referred to as ION), the Department of Surgery, Liverpool University (‘LIV’), and the CNIO Madrid, Spain (‘SPA’), (in total 371 urines) for validation purposes. Urine samples for patients with other benign and malignant hepatobiliary pathologies (n=117) were obtained from LIV. All samples were collected with full ethical approval from the involved centres, and with informed consent from all individuals who donated urine samples. The specimens in all participating centres were collected using the same standard operating procedures: clean-catch, midstream urine was collected, frozen within 2 hours of collection and stored at −80° C. until utilized. Of importance, all the samples were derived from patients with no history of renal diseases; dipstick test analysis (Bayer multistix SG 08935414) was also performed to exclude potential bilirubinemia, proteinuria, bacterial contamination and hematuria. Samples were collected before surgery or chemotherapeutic treatment. Matching plasma samples to measure CA19.9 were available from RLH and LIV.

GeLC-MS/MS Analysis of Urine Proteomes

Six urine samples (three males and three females) for each group (H, CP and PDAC; in total 18 samples) were utilised: H males/females age 45, 50, 60/44, 45, 54 years; CP males/females age 46, 48, 51/47, 69, 74 years; PDAC males/females age 44, 74, 84/71, 73, 77 years; male PDAC stage all IIB/female two IB, one IIA. All urine samples were desalted and concentrated as described previously (Weeks M E et al. “Analysis of the urine proteome in patients with pancreatic ductal adenocarcinoma”, Proteomics Clinical Applications 2008; 2:1047-57). 20 μg of each pre-processed pool of three samples per group were separated in duplicate on 4-12% mini-gels (Invitrogen); female and male urines were analysed separately. The gels were stained with Colloidal Coomassie, and each sample lane cut using a grid into 40 equally sized slices. Gel slices were digested robotically with trypsin and resultant peptides analysed by nano LC/MS/MS using a nanoAcquity (Waters) interfaced to a LTQ Orbitrap XL tandem mass spectrometer (ThermoFisher). Product ion data were searched against the human IPI protein database using Mascot, and subsequently parsed into the Scaffold software (Proteome Software) for collation into non-redundant protein lists. Reversed database searching was used to assess false discovery rates, the target protein FDR being <0.5% per sample. A semi-quantitative assessment of relative protein abundance between PDAC, CP and Healthy samples was obtained by using the spectral counting approach (Liu H et al., “A model for random sampling and estimation of relative protein abundance in shotgun proteomics”, Analytical Chemistry, 2004; 76:4193-201).

### Urine Biomarkers and CA19.9 Measurements

Total protein concentration in urines was determined by Bradford assay (Coomassie Protein Assay Reagent, Pierce). The quantitative determination of human LYVE-1 (Cat# SEB049Hu, Uscn Life Science Inc.) and human TFF1 (Cat# ELH-LYVE1-001, RayBiotech, Inc.) was performed according to the manufacturer's instructions; human ReG1A levels were initially assessed in our laboratory, and afterwards by BioVendor Analytical Testing Service (BioVendor—Laboratorní medicína a.$). Calibration curves were prepared using purified standards for each protein assessed. Curve fitting was accomplished by a four-parameter logistic regression following the manufacturer's instructions. The limits of detection and the coefficient of variation (CV) for each of the ELISA assays were as follows: LYVE—1-8.19 pg/ml, intra-assay CV—9%, inter-assay CV—12%, TFF1—0.037 ng/ml, intra-assay CV—9%, inter-assay CV—12%. REG1A—0.094 ng/ml, intra-assay CV—9%, inter-assay CV 20%; REG1B-3.13 pg/ml, intra-assay CV—3.9%, inter-assay CV—2.7%. Urine creatinine was measured by the Jaffé method using the Roche Cobas 8000 system (Roche Diagnostics, Mannheim, Germany) at the Clinical Biochemistry Laboratory, RLH (London, UK). Levels of Ca19.9 in plasma and urine were measured at the Clinical Biochemistry Laboratory, RLH using a Roche Modular E170 instrument according to the routine protocols.

### Tissue Microarrays and Immunohistochemistry (IHC)

The details of the tissue microarray and scoring procedure used in evaluating the expression of the biomarkers was described previously (Ene-Obong A et al., “Activated pancreatic stellate cells sequester CD8+ T cells to reduce their infiltration of the juxtatumoral compartment of pancreatic ductal adenocarcinoma”, Gastroenterology, 2013; 145:1121-32). IHC was performed with anti-REG1A (Abcam, Rabbit polyclonal, ab47099, 1:100 dilution), anti-TFF1 (Abcam, Rabbit polyclonal, ab50806, 1:100 dilution), and anti-LYVE1 (Acris, Rabbit polyclonal, DP3500PS, 1:100 dilution) antibodies using the Ventana Discovery system, according to standard protocols (sCC1, 1 h incubation).

### Statistical Analysis

To identify potential urine biomarkers from the MS data, the statistical analysis was performed on the normalized data (based on the sum of spectral counts/sample) using Arraytrack software (http;edkb.fda.go/webstart/arraytrack) and a t-test. The data were further filtered according to both p values and fold change between any two sample groups.

The concentrations of the selected proteins (LYVE1, REG1A and TFF1) were subsequently explored using ELISA assays, and the obtained results analysed using nonparametric Kruskal-Wallis test followed by Dunn's post test with GrafPadPrism. Correlation between the three biomarkers was assessed using Pearson's correlation coefficient.

Each individual biomarker and the panel were investigated for their ability to discriminate between PDAC patients (all stages, or early stages I-II) and control samples (healthy and CP) using ROC analysis and an hold-out approach. For each comparison, 70% of the subjects in the patient and control datasets were randomly selected for inclusion in the training dataset. Logistic regression was then applied. All protein concentration data were natural log-transformed and mean-centered prior to regression analysis. In individual biomarker analyses, creatinine-normalised data were used to correct for the urine dilution factor; for the panel analysis, the model included the three biomarkers (prior to creatinine normalisation) and was adjusted for creatinine and age (as the median age of PDAC patients was higher than that of healthy and CP individuals, Table 4), i.e. 5-parameter model. Separate models were applied to the training datasets for the comparison of PDAC all stages versus healthy, PDAC stages I-II versus healthy, PDAC all stages versus CP and PDAC stages I-II versus CP. ROC curves were generated for each of the above regression models; the area under the curve (AUC), and the sensitivity (SN) and specificity (SP) at the ‘optimal’ cut-point for discrimination between groups were obtained. The optimal cut-point corresponded to the point closest to the top-left part of the plot in the ROC plane (coordinates 0,1) with optimal SN and SP according to the following criterion:

min((1−sensitivities)²+(1−specificities)²),

as calculated by the ‘ci.threshold’ procedure of the R ‘pROC’ package (Robin X et al., “pROC: an open-source package for R and S+ to analyse and compare ROC curves”, BMC Bioinformatics, 2011; 12:77). This approach been showed to have good performance in the estimation of the optimal cut-point of a biomarker.

The rest of the subjects (30%) formed independent datasets which were used for model validation. For the primary analysis (all PDAC versus Healthy), 49 PDAC and 28 healthy samples give more than 90% power to detect a standardized difference of 1.0 (i.e. a difference between PDAC and healthy samples of at least one standard deviation) using a one-sided test.

Validation was performed by classifying each sample in the validation dataset according to the logistic regression model developed based on the training dataset, and comparing this classification with the actual diagnosis, hence deriving a new ROC curve. The optimal cut-points computed for the training sets were used to derive the SN and SP of the validation dataset. Confidence intervals (CI, 95%) for AUCs were derived based on DeLong′ asymptotically exact method to evaluate the uncertainty of an AUC (DeLong E R et al., “Comparing the areas under two or more correlated receiver operating characteristic curves: a nonparametric approach”, Biometrics, 1988; 44:837-45).); SN and SP, 95% CI were derived using non-parametric stratified resampling with the percentile method (2,000 bootstrap replicates) as described by Carpenter J & Bithell J., “Bootstrap confidence intervals: when, which, what? A practical guide for medical statisticians”, Stat Med, 2000; 19:1141-64.). AUCs were compared using DeLong's 1-sided test for correlated/paired AUCs).

For exploratory analyses, ROC curves were derived for the comparison of PDAC stage I-IIA versus healthy or CP based on logistic regression modelling using all available samples.

ROC curve analyses were performed in R version 2.13.0 (The R Foundation for Statistical Computing, http://www.r-project.org/foundation) using procedures from the Epi, pROC and ROCR (Sing T et al., “ROCR: visualizing classifier performance in R.”, Bioinformatics, 2005; 21:3940-1) packages.

### Urine Proteomes

An in-depth proteomics analysis by GeLC/MS/MS of 18 urine specimens derived from PDAC, CP and healthy (H) individuals (6 per group, three males, three females) (FIG. 9A) was undertaken. This analysis resulted in the identification of around 1,500 (1,198 in male and 1,061 in female urine) non-redundant proteins. These proteins originated from all cellular compartments and were mapped using IPA (Ingenuity pathway analysis, http://www.ingenuity.com/) to a number of cellular functions and diseases, confirming that urine provides a rich source of diverse proteins with respect to their origin and functional roles.

The MS analysis was performed separately on urine samples from male and female subjects. Considerable gender-specific differences were noticed: of 997 proteins identified in healthy urine samples, 398 (40%) were unique to male urines, 118 (12%) were unique to female and 481 (48%) were common to both.

Among around 200 differentially expressed between the three experimental groups (PDAC, CP, H) three proteins commonly deregulated in both males and females: LYVE1, REG1A and TFF1, were selected for further evaluation based on the statistics (p-values, fold change), interrogation of both Pancreatic Expression Database (PED) (http://www.pancreasexpression.org/) and additional literature search to for previous knowledge on the potential candidates, and also on the availability of commercial ELISA assays. While REG1B in the proteomics data appeared to be slightly better candidate, only REG1A ELISA assay was available commercially at the time. However, when REG1B ELISA became available, a subset of urine samples was tested and similar results were obtained (see later). The presence of the three selected biomarkers as full-size proteins: 35 kDa for LYVE1, 19 kDa for REG1A, and 9 kDa for TFF1 in urine specimens was confirmed by Western blot examination (data not shown).

### Biomarker Panel in Detecting PDAC

The selected biomarkers were subsequently assessed using ELISA assays on 371 urine samples collected from three centres: London and Liverpool, U K, and Madrid, Spain. Demographics and clinical characteristics of patients and healthy participants included in the study are shown in Table 4 (below).

TABLE 4
Demographics and clinical characteristics of the healthy and patient cohorts

| | Normal | | | CP | | | PDAC | | | |
|---|---|---|---|---|---|---|---|---|---|---|
| | | | Age range | | | Age range | | | Age range | Stage/n |
| | Cases (n) | Gender | (Median) | Cases (n) | Gender | (Median) | Cases (n) | Gender | (Median) | plasma |
| LON | 87 | M = 46 | 28-87 | 45 | M = 32 | 29-82 | 60 | M = 38 | 29-82 | I = 4/4 |
| | | F = 41 | (55) | | F = 13 | (53) | | F = 22 | (64) | IIA = 1/1 |
| | | | | | | | | | | IIB = 13/13 |
| | | | | | | | | | | III = 33/30 |
| | | | | | | | | | | IV = 6/5 |
| | | | | | | | | | | U = 3/3 |
| Plasma | 28 | M = 16 | 28-67 | 19 | M = 14 | 29-74 | 56 | M = 34 | 29-82 | |
| (CA19.9) | | F = 12 | (46) | | F = 5 | (54) | | F = 22 | (64) | |
| LIV | 0 | N/A | N/A | 41 | M = 25 | 29-82 | 91 | M = 53 | 39-83 | I = 3/3 |
| | | | | | F = 16 | (51) | | F = 38 | (68) | IIA = 8/8 |
| | | | | | | | | | | IIB = 42/42 |
| | | | | | | | | | | III = 38/38 |
| | | | | | | | | | | IV = 0/0 |
| | | | | | | | | | | U = 0/0 |
| Plasma | 0 | N/A | N/A | 31 | M = 17 | 37-73 | 91 | M = 53 | 39-83 | |
| (CA19.9) | | | | | F = 14 | (51) | | F = 38 | (68) | |
| SPA | 0 | N/A | N/A | 6 | M = 4 | 54-68 | 41 | M = 23 | 43-94 | I = 0/NA |
| | | | | | F = 2 | (57) | | F = 18 | (72) | II = 0/NA |
| | | | | | | | | | | III = 0/NA |
| | | | | | | | | | | IV = 0/NA |
| | | | | | | | | | | U = 41/NA |
| Plasma | 0 | N/A | N/A | 0 | N/A | N/A | 0 | N/A | N/A | |
| (CA19.9) | | | | | | | | | | |
| Total | 87 | M = 46 | 28-87 | 92 | M = 61 | 29-82 | 192 | M = 114 | 29-94 | I = 7/7 |
| | | F = 41 | (55) | | F = 31 | (54) | | F = 78 | (68) | IIA = 9/9 |
| | | | | | | | | | | IIB = 55/55 |
| | | | | | | | | | | III = 71/68 |
| | | | | | | | | | | IV = 6/5 |
| | | | | | | | | | | U = 44/3 |
| Plasma | 28 | M = 16 | 28-67 | | M = 31 | 29-74 | | M = 87 | 29-83 | |
| (CA19.9) | | F = 12 | (46) | 50 | F = 19 | (53) | 147 | F = 60 | (67) | |

### PDAC Stage I-IV Versus Healthy

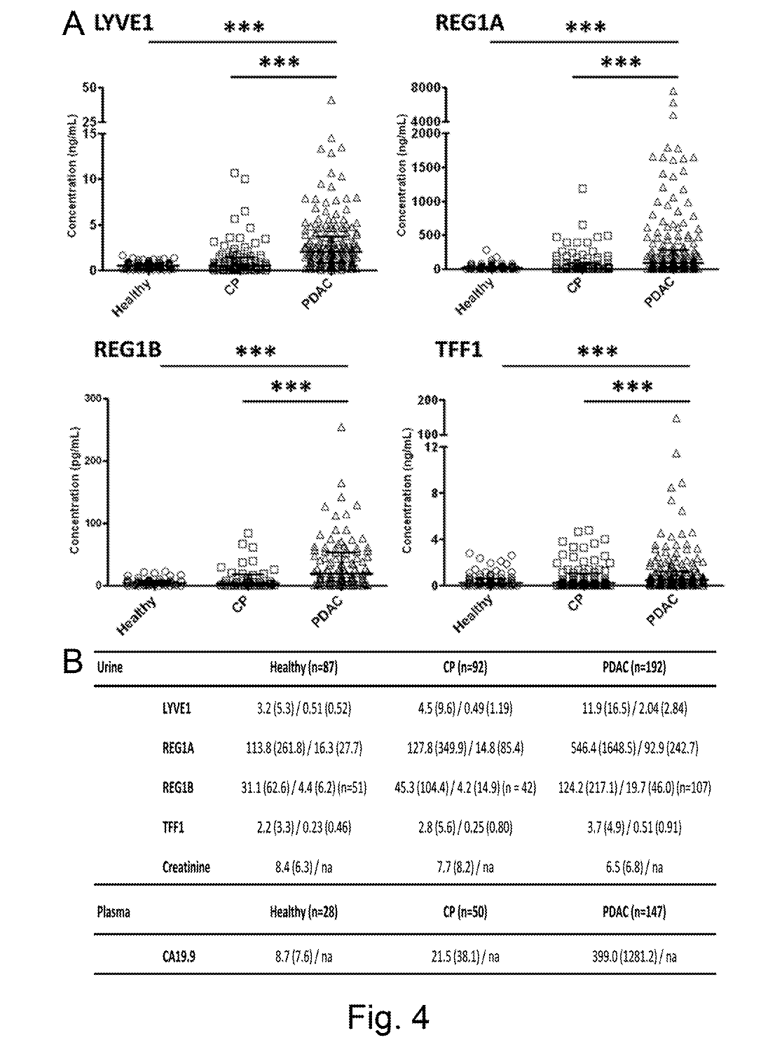

The ELISA analysis showed significantly higher urine concentrations of each of the candidate biomarkers in the urine of PDAC patients (n=192) when compared to healthy samples (n=87, all with p<0.0001, FIG. 4). Of note, REG1B and REG1A ELISA assays produced similar results (FIG. 4).

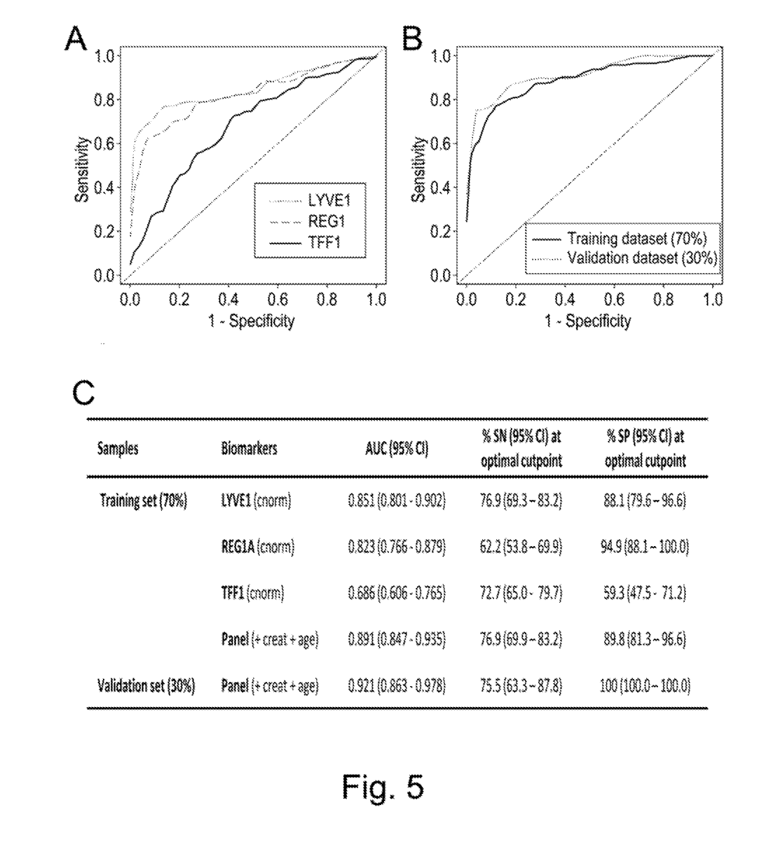

In PDAC, LYVE1, REG1A and TFF1 were positively correlated with each other, while in healthy samples, only LYVE1 and REG1A were correlated (FIG. 10). The diagnostic performance of LYVE1, REG1A and TFF1 was established using Receiver Operating Characteristic (ROC) curve analysis (FIG. 5). Their individual performance in discriminating between PDAC stage I-IV and healthy urines was assessed first, in a training dataset (70% of the samples, n=143 and n=59, respectively). Individual (creatinine-normalised) urine biomarkers were able to discriminate between the two groups with AUC values of 0.851 (95% CI 0.801-0.902) for LYVE1, 0.823 (95% CI 0.766-0.879) for REG1 and 0.686 (95% CI 0.606-0.765) for TFF1, with respective SN of 76.9% (95% CI 69.3-83.2), 62.2% (95% CI 53.8-69.9) and 72.7% (95% CI 65.0-79.7), and respective SP of 88.1% (95% CI 79.6-96.6), 94.9% (95% CI 88.1-100.0), and 59.3% (95% CI 47.5-71.2) (FIG. 5A, C). The three biomarkers were then combined into a panel adjusted for creatinine and age (FIG. 5B). The results of the logistic regression model underlying the ROC analysis in the training and validation (30% of the samples, PDAC n=49, healthy n=28) datasets are shown in FIGS. 5B and C. The panel achieved SN>75% and SP>85% for AUCs of 0.891 (95% CI 0.847-0.935) and 0.921 (95% CI 0.863-0.978) in the training and validation datasets, respectively, thus showing better performance than any of the individual biomarkers.

### PDAC Early Stages Versus Healthy

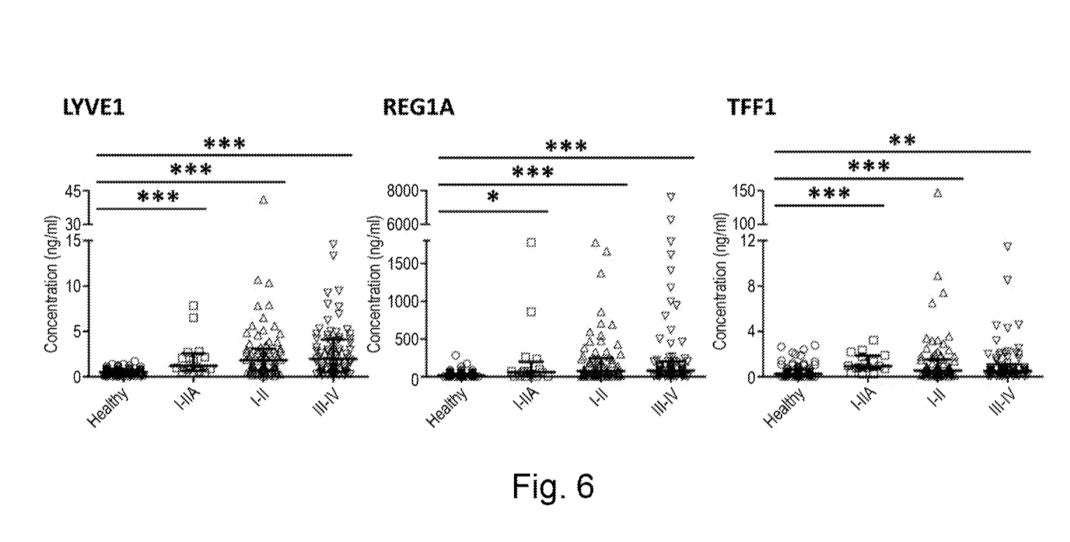

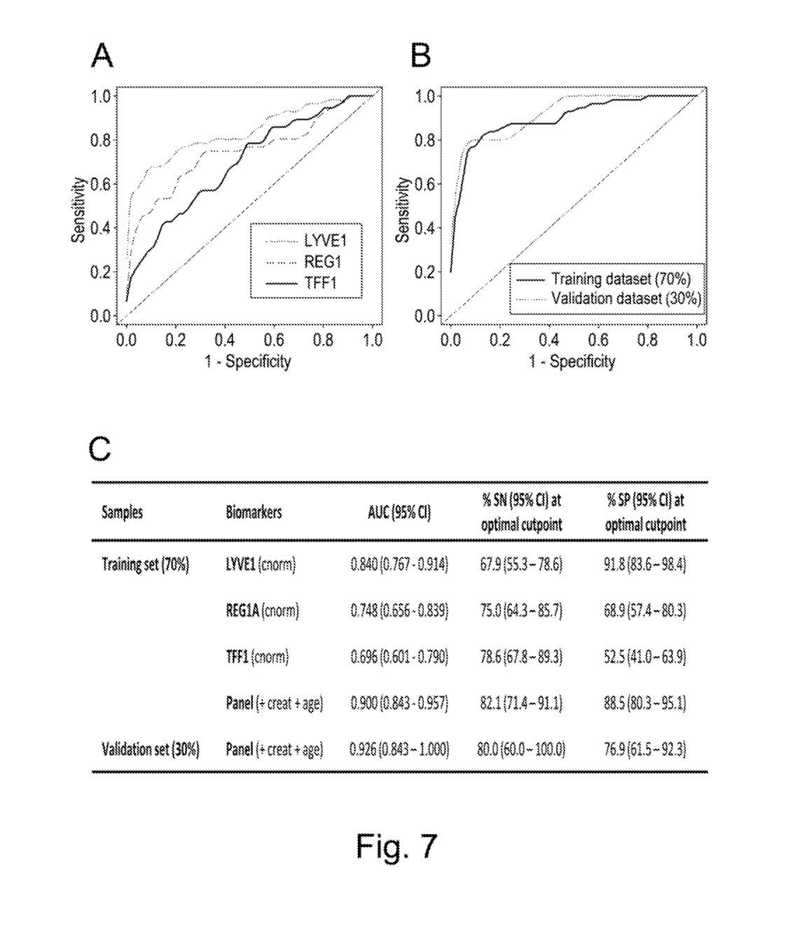

Next, the performance of the biomarkers in discriminating early stage cancers from healthy individuals was assessed. Tumour staging information was available for 148 (77%) of the PDAC patients. The concentrations of each of the biomarkers were significantly increased in later stages (stage n=77, all p<0.001), in stages I-II (n=71, all p<0.001) and in stages I-IIA (locally invasive disease without lymph node metastases, n=16, p<0.05) compared to healthy people (n=87) (FIG. 6). The concentrations of LYVE1 and TFF1 were also higher in stage I cancers (p<0.001 and p<0.05, respectively; data not shown). As a limited number of stage I urine samples was available (n=7), the diagnostic accuracy of the urine markers on combined PDAC stage I-II data was assessed. The performance of the individual markers and the panel in discriminating between PDAC stage I-II from healthy urines was first assessed in a new training dataset (70% of the samples; PDAC stage I-II n=56 and healthy n=61, respectively). A new 5-parameter model was built using this training dataset and validated using the rest of the data (30% of the samples; PDAC stage I-II n=15, Healthy n=26) (FIG. 7A, B). The panel achieved AUCs of 0.900 (95% CI 0.843-0.957) and 0.926 (95% CI 0.843-1.000) in the training and validation datasets, respectively (FIG. 7C). Therefore, the urine biomarker panel can differentiate early PDAC from healthy samples with high accuracy.

As an exploratory analysis, the urine samples from individuals for which matched plasma samples were available were selected so CA19.9 values could be obtained. The ROC curves were derived for plasma CA19.9 (as a categorical variable with a cut-off at clinically established threshold of 37 U/mL), the panel, and a combination of the panel and CA19.9. For the comparison of PDAC stage I-II (n=71) versus healthy (n=28) samples, AUCs of 0.880 (95% CI 0.947-0.999) for CA19.9, and 0.973 (95% CI 0.947-0.999) were obtained for the panel, which was significantly greater than plasma CA19.9 alone (p=0.005). The addition of plasma CA19.9 to the panel significantly increased the AUC to 0.991 (95% CI 0.979-1.000, p=0.04, FIG. 8A/C). When PDAC stage I-IIA (n=16) were compared to healthy samples, AUCs were 0.839 (95% CI 0.719-0.959) for CA19.9, and 0.971 (95% CI 0.929-1.000) for the panel (p=0.006). The addition of plasma CA19.9 to the panel did not result in any improvement (AUC=0.969, 95% CI 0.924-1.000, p=0.7, FIG. 8B/C).

Biomarker Panel in Differentiating PDAC from CP

The ability of the biomarker panel in differentiating PDAC from CP was assessed.

### PDAC Stage I-IV Versus CP

Urine concentration for all three biomarkers was higher in PDAC (n=192) compared to CP samples (n=92), all with p<0.001, (FIG. 3) and as for PDAC, the biomarker concentrations were positively correlated with each other in the CP data (FIG. 10). In the training dataset (PDAC n=143, CP n=62) LYVE1 and REG1 were able to discriminate between the two groups with SN of 77-78% and SP of 66-69% (respective AUC values of 0.775 (95% CI 0.704-0.846) and 0.722 (95% CI 0.643-0.801, FIG. 11), while the SP of TFF1 only reached 50% for a similar SN. Combining the three biomarkers into a panel only improved marginally the performance of LYVE1 and REG1 alone as assessed in the training (AUC=0.815, 95% CI 0.752-0.878), and validation (PDAC n=49, CP n=30, AUC=0.839, 95% CI 0.751-0.928) datasets.

### PDAC Early Stages Versus CP

Biomarker urine concentrations were significantly increased in stages I-II PDAC (n=71) compared to CP (n=87), with p<0.001 for each of the three biomarkers (data not shown). The panel achieved high SN (>85%) in both the training (PDAC stage I-II n=56, CP n=66) and validation (PDAC stage I-II n=15, CP n=26) datasets, but relatively low SP (66.7% and 50%), similar to the SP observed for individual biomarkers, with respective AUCs of 0.831 (95% CI 0.762-0.901) and 0.846 (95% CI 0.730-0.963, FIGS. 11D-F).

As before, the panel was explored in combination with plasma CA19.9. For the comparison of PDAC stage I-II (n=71) versus CP (n=50) samples, the ROC curves showed AUCs of 0.775 (95% CI 0.699-0.852) for CA19.9, 0.830 (95% CI 0.759-0.902) for the panel (p=0.1), and 0.885 (95% CI 0.825-0.945) for the panel in combination with CA19.9 (p=0.01 for superiority over the panel alone) (FIGS. 12A/C). In the comparison of PDAC stage I-IIA (n=16) versus CP, the ROC curves showed AUCs of 0.735 (95% CI 0.609-0.861) for CA19.9, 0.871 (95% CI 0.770-0.972) for the panel (p=0.004 for superiority over plasma CA19.9), and 0.866 (95% CI 0.749-0.984) for the combination (p=0.6) (FIG. 12B/C). Therefore, the panel performed better in differentiating stage I-IIA from CP than CA19.9.

Refer also to the table below and corresponding FIG. 15, demonstrating the ability of each of miR-26a, miR-30a, miR-30b and miR-106b to distinguish between CP and healthy samples. The data demonstrate that each of these biomarkers had significant differential expression in CP versus healthy tissues. Furthermore, miR-30b and miR106b were elevated in CP when compared with PDAC.

The additional four miRNAs: miR-26a, miR-30a, miR-30b, and miR-106b, were found differentially expressed using Affymetrix chips (as described for the previously reported 4 miRNAs), in particularly between healthy individuals and patients with chronic pancreatitis (CP).

Briefly, global low molecular weight (LMW) RNA expression profile was determined using the Affymetrix GeneChip microRNA v. 3.0 Arrays, interrogating 26 urine samples, which included 13 PDAC samples (four Stage I, three Stage II, six Stages III-IV), six CP and seven healthy individuals. For one of the healthy samples, two biological replicates (independent RNA extractions) and two technical replicates were also performed. Therefore, in total, 30 arrays were interrogated. (Microarray data are deposited in GEO, accession number GSE71962).

ANOVA applied to identify differentially expressed miRNAs between the sample groups (H, CP and PDAC Stage I and Stages II-IV), led to the identification of 79 statistically significant differentially expressed miRNAs (FDR<0.05), including the four miRNAs mentioned above. All four miRNAs were over-expressed in CP when compared to healthy individuals. Furthermore, miR-30b and miR-106b were elevated in CP when compared with PDAC (FIG. 1 and Table 1). The sequence of the 4 miRNAs is indicated in Table 2.

Of the four new miRNAs, miR-26a and miR-30b (as well as miR-204) have recently been described in high grade-invasive cystic lesions (Wang J. et al., Cancer Letters 2015, 356: 404-409), and miR-30a, miR-30b and miR-106b were able to predict the risk of malignant transformation in IPMN (Matthaei H. et al., Clinical Cancer Res 2012, 18:4713-4724). However, urine biomarkers have not been reported previously.

| | CP v H | p-value | CP v St I | p-value | CP v St II-IV | p-value |
|---|---|---|---|---|---|---|
| miR-26a | Up [8.4x] | 0.00267 | | ns | | ns |
| miR-30a | Up [10x] | 0.00660 | | ns | | ns |
| miR-30b | Up [3.3x] | 0.00008 | Up [2.1] | 0.02621 | Up [3.0x] | 0.00042 |
| miR-106b | Up [2.4x] | 0.00354 | Up [2.1] | 0.04403 | Up [2.2x] | 0.01002 |

### Biomarker Expression in Urine of Other Hepatobiliary Pathologies

Finally, the expression of the biomarkers in urine specimens collected from patients with several other benign or malignant hepatobiliary pathologies was explored and compared to the expression in patients with early stage PDAC (FIG. 13). Urine levels of LYVE1 in PDAC stage I-II samples were higher than in IPMNs, AMP and pancreatic NETs specimens; REG1A levels were only significantly higher in early stage PDAC compared to IPMNs. Plasma CA19.9 levels were significantly higher in PDACs stage I-II compared to pancreatic NETs and DuCA samples. This might suggest a potential utility for LYVE1 and REG1A in distinguishing other benign or malignant hepatobiliary pathologies from early stage PDACs.

### Tissue Origin of the Three Biomarkers

Having demonstrated a good performance of the panel in differentiating early cancer patients from healthy individuals, it was next sought to establish the expression of the biomarkers in pancreatic tissue. Immunohistochemistry (IHC) was performed using in-house constructed PDAC tissue microarrays. A strong expression of REG1A was seen in histologically normal adjacent acinar cells, but the staining was also seen in 44/60 tumours (73%) (FIG. 14A). TFF1 was absent in normal pancreas, but was expressed in 43/60 (72%) of PDACs (FIG. 14B). While no LYVE1 expression was seen in any of the cancer cells, it was seen in scarce lymphatic vessels in eight PDAC tissues (FIG. 14C). Next, the levels of all three biomarkers was measured in urines from seven PDAC patients for whom samples were collected prior to and after surgery (FIG. 14D). In all patients, levels of LYVE1 and REG1A decreased after surgery, and this was also seen in six out of seven patients for TFF1 (except for Patient 1, where the first post-surgical urine sample was collected four months after the procedure), likely due to substantial loss of tumour mass after surgery.

Finally, as several reports have indicated that CA19.9 is also present in urine and can be used for cancer diagnosis, and was even superior to blood CA19.9 in some cases, Ca19.9 levels in urine samples were measured and compared them to matched plasma CA19.9. Urine CA19.9 did not prove useful in differentiating PDAC from CP and healthy urines in this case (data not shown).

The present inventors have identified a number of new biomarkers useful in the diagnosis of chronic pancreatitis and pancreatic cancer at its various stages of development. The inventors have demonstrated at least that:
miR-143 can be used on its own or in combination with miR-30e and/or miR-223 in the diagnosis of early stage PDAC.
A panel comprising miR-143, miR-223 and/or miR-204 can distinguish between early stage PDAC and later stage PDAC.
A panel comprising miR-223 and/or miR-204 can distinguish between early stage PDAC and chronic pancreatitis (CP).
miR-30b and miR106b can also be used (individually or separately) to diagnosis PDAC, and also to distinguish between early stage PDAC and CP, and between late stage PDAC and CP.
Each of miR-26a, miR-30a, miR-30b and miR-106b can distinguish between CP and healthy patients.
Each of miR-26a, miR-30a, miR-30b and miR-106b can predict the progression of pancreatic disease from IPMNs (cysts) to malignant PDAC.

PDAC is one of the most challenging cancers to detect; the majority of patients thus present at an advanced stage of the disease. Hence less than 20% of PDAC patients undergo potentially curative surgery, while the remainder can only be offered palliative treatment. Here, a three-biomarker urine panel is described that discriminates early stage PDAC patients from healthy subjects with high accuracy. A diagnostic test based on urine specimens was developed as this body fluid has several advantages over blood: it is far less complex, provides an ‘inert’ and stable matrix for analysis, and can be repeatedly and non-invasively sampled in sufficient volumes. So far, more than 2,300 proteins have been detected in urine, of which at least a third are of a systemic origin. As an ultrafiltrate of blood, it can be expected that at least some of the biomarkers will be found in higher concentration in urine than in blood.

When combined, REG1A and TFF1, LYVE1 form a powerful urinary panel that can detect patients with stages I-II PDAC, with over 90% accuracy. The exploratory analyses suggest that when combined with CA19.9, accuracy may be increased. In addition, the panel may prove useful in discriminating patient in stages I-IIA from healthy ones.

Being completely non-invasive and inexpensive, this urine screening test could, when coupled with timely surgical intervention, lead to a much improved outcome in patients with high-risk of developing pancreatic adenocarcinoma.

1. A method of diagnosing or testing for pancreatic ductal adenocarcinoma (PDAC) comprising determining a level of expression or concentration of one or more miRNAs selected from a group consisting of miR-143, miR-223, miR-204, miR-30e, miR-26a, miR-30a, miR-30b, miR-106b, or a combination thereof, in a biological sample.

2. The method according to claim 1, wherein the method comprises detecting the level of expression or concentration of miR-143 in the biological sample.

3. The method according to claim 1, wherein the method comprises detecting the level of expression or concentration of miR-143 and miR-30e in the biological sample.

4. The method according to claim 1, wherein the method comprises detecting the level of expression or concentration of miR-143, miR-223 and miR-30e in the biological sample.

5. The method according to claim 2, wherein the method provides differential diagnosis distinguishing between stage I PDAC and a healthy patient.

6. The method according to claim 1, wherein the method comprises detecting the level of expression or concentration of miR-223 and miR-204 in the biological sample.

7. The method according to claim 1, wherein the method comprises detecting the level of expression or concentration of miR-30b and miR-106b in the biological sample.

8. The method according to claim 1, wherein the method comprises detecting the level of expression or concentration of at least one, at least 2, or at least 3 of miR-223, miR-204, miR-30b an miR-106b in the biological sample.

9. The method of claim 1, wherein the method provides differential diagnosis distinguishing between PDAC and chronic pancreatitis (CP).

10. The method of claim 9, wherein the PDAC is stage I PDAC.

11. The method of claim 1, wherein the method provides a differential diagnosis distinguishing between stage II to IV PDAC and chronic pancreatitis (CP).

12. The method according to claim 1, wherein the method comprises detecting the level of expression or concentration of miR-143, miR-223 and miR-204 in the biological sample.

13. The method of 12, wherein the method is a differential diagnosis distinguishing between stage I PDAC and a later stage of PDAC.

14. The method according to claim 1, wherein determining the level of expression or concentration comprises contacting the same with a binding molecule or binding molecules specific for the one or more miRNAs being analyzed.

15. The method according to claim 14 wherein the binding molecule is a nucleic acid.

16. The method according to claim 1, wherein the biological sample is a urine, saliva, whole blood, serum or pancreatic tissue sample.

17. The method according to claim 1, wherein the sample is from a human.

18. The method according to claim 1, wherein the PDAC is stage I PDAC.

19. The method of claim 1, further comprising a step of quantifying the expression level of the miRNA or miRNAs.

20. The method of claim 19, wherein quantification of the expression level of the miRNA or miRNAs comprises the use of real-time quantitative PCR, microarray analysis, RNA sequencing, Northern blot analysis, in situ hybridisation, nCounter Analysis system analysis, or Integrated Comprehensive Droplet Digital Detection (IC 3D) analysis.

21. The method of claim 1, wherein the biological sample is processed prior to determination of the level of expression or concentration of the one or more miRNAs in the biological sample.

22. The method of claim 21, wherein the biological sample is enriched for miRNA.

23. The method of claim 21, wherein the one or more miRNAs are extracted from the biological sample.

24. The method of claim 23, wherein the miRNA extraction step comprises chemical extraction, or solid-phase extraction.

25. The method of claim 24, wherein the solid-phase extraction is chromatographic extraction.

26. The method of claim 19, wherein the step of quantification of the expression level of the miRNA or miRNAs comprises RNA sequencing.

27. The method of claim 19, wherein the one or more miRNAs are translated into one or more cDNAs prior to quantification.

28. The method of claim 19, wherein the step of quantification of the expression level of the miRNA or miRNAs is achieved using a microarray.

29. The method of claim 28, comprising the steps of capturing the one or more microarrays on a solid support and detecting hybridization.

30. The method of claim 29, further comprising sequencing the one or more miRNA or cDNA molecules.

31. The method of claim 1, further comprising a step of comparing the level of expression or concentration of one or more miRNAs with a reference.

32. The method of claim 31, wherein the reference is a biological sample from a healthy patient.

33. The method of claim 1, wherein the biological sample is from a patient having or suspected of having PDAC.

34. The method of claim 1, further comprising determining a level of expression or concentration of one or more proteins selected from a group consisting of LYVE1, REG1 and TFF1.

35. The method of claim 34, further comprising determining the level of expression or concentration of CA19.9 protein.

36. The method of claim 34, wherein the level of expression or concentration of the one or more miRNAs and the level of expression or concentration of the one or more proteins is determined in the same biological sample.

37. The method of claim 34, wherein the level of expression or concentration of the one or more miRNAs and the level of expression or concentration of the one or more proteins is determined in a different biological sample from the same patient.

38. The method of claim 34, wherein the level of expression or concentration of the one or more miRNAs and the level of expression or concentration of the one or more proteins is determined simultaneously.

39. The method of claim 34, wherein determining the level of expression or concentration of one or more proteins comprises contacting a sample with a binding molecule or binding molecules specific for the one or more proteins being analyzed.

40. The method according to claim 39 wherein the binding molecule is an antibody, an antibody fragment, a protein or an aptamer.

41. A method of treating PDAC, comprising diagnosing a patient as having or as being suspected of having PDAC using a method as defined in claim 1, and administering to the patient a therapy for PDAC.

42. A method of treating PDAC in a patient, wherein the patient has been determined as having PDAC or as being suspected of having PDAC according to a method as defined in claim 1, comprising administering to the patient a therapy for PDAC.

43. The method of claim 41, wherein the therapy for PDAC comprises chemotherapy and/or radiotherapy.

44. The method of claim 43, wherein the chemotherapy comprises administration of gemcitabine, fluorouracil (5-FU), irinotecan, oxaliplatin and/or folinic acid (leucovorin).

45. The method of claim 41, wherein the therapy for PDAC comprises resection of the pancreatic duct or resection of a PDAC tumour.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. A kit for testing for PDAC or CP comprising a means for measuring the level of expression or concentration of one or more miRNAs selected from the group consisting of miR-143, miR-223, miR-204, miR-30e, miR-26a, miR-30a, miR-30b and miR-106b or combinations thereof, in a biological sample.

68. The kit of claim 67, comprising a means for measuring the level of expression or concentration of at least two of the miRNAs.

69. The kit of claim 67, comprising a means for measuring the level of expression or concentration of at least three of the miRNAs.

70. The kit of claim 67 comprising a means for detecting the expression of miR-143, miR-223 and miR-30e.

71. The kit of claim 67 comprising a means for detecting the expression of miR-143, miR-223 and miR 204.

72. The kit of claim 67, wherein the means for detecting is a biosensor or specific binding molecule.

73. The kit of claim 72, wherein the biosensor is an electrochemical, electronic, piezoelectric, gravimetric, pyroelectric biosensor, ion channel switch, evanescent wave, surface plasmon resonance or biological biosensor

74. The kit of claim 67, wherein the means for detecting comprises a dipstick coated with a membrane, wherein the dipstick further comprises:
(a) a first section to which is bound an unlabeled binding molecule with specific affinity for the miRNA whose expression is being detected;
(b) a second section that is blocked with a non-reactive miRNA; and, optionally,
(c) a third section to which is bound the miRNA whose expression is being detected.

75. The kit of claim 67, wherein the kit further comprises one or more vessels for containing the biological sample.

76. The kit of claim 67, wherein the means for detecting the amount of expression or concentration of the one or more miRNAs is a microarray.

77. The kit of claim 76, wherein the microarray comprises specific binding molecules that hybridise to miR-143, miR-223 and miR-204.

78. The kit of claim 76, wherein the microarray comprises specific binding molecules that hybridise to miR-143, miR-223 and miR-30e.

79. The kit of claim 76, wherein the microarray comprises specific binding molecules that hybridise to miR-143 and miR-30e

80. The kit of claim 76, wherein the microarray comprises specific binding molecules that hybridise to miR-223 and miR-204.

81. The kit of claim 67, wherein the kit further comprises means for detecting the amount of expression or concentration of one or more proteins selected from the group consisting of LYVE1, REG1, TFF1 and CA19.9.

82. The kit of claim 81, wherein the kit further comprises one or more solvents for extracting the protein and/or miRNAs from the biological sample.


SEQUENCE LISTING (12 sequences)

SEQ ID NO: 1 - protein, 322 aa, Human
MARCFSLVLLLTSIWTTRLLVQGSLRAEELSIQVSCRIMGITLVSKKANQQLNFTEAKEACRLLGLSLAGKDQVETALKASFETCSYGWVGDGFVVISRISPNPKCGKNGVGVLIWKVPVSRQFAAYCYNSSDTWTNSCIPEIITTKDPIFNTQTATQTTEFIVSDSTYSVASPYSTIPAPTTTPPAPASTSIPRRKKLICVTEVFMETSTMSTETEPFVENKAAFKNEAAGFGGVPTALLVLALLFFGAAAGLGFCYVKRYVKAFPFTNKNQQKEMIETKVVKEEKANDSNPNEESKKTDKNPEESKSPSKTTVRCLEAEV

SEQ ID NO: 2 - protein, 166 aa, Human
MAQTSSYFMLISCLMFLSQSQGQEAQTELPQARISCPEGTNAYRSYCYYFNEDRETWVDADLYCQNMNSGNLVSVLTQAEGAFVASLIKESGTDDFNVWIGLHDPKKNRRWHWSSGSLVSYKSWGIGAPSSVNPGYCVSLTSSTGFQKWKDVPCEDKFSFVCKFKN

SEQ ID NO: 3 - protein, 166 aa, Human
MAQTNSFFMLISSLMFLSLSQGQESQTELPNPRISCPEGTNAYRSYCYYFNEDPETWVDADLYCQNMNSGNLVSVLTQAEGAFVASLIKESSTDDSNVWIGLHDPKKNRRWHWSSGSLVSYKSWDTGSPSSANAGYCASLTSCSGFKKWKDESCEKKFSFVCKFKN

SEQ ID NO: 4 - protein, 84 aa, Human
MATMENKVICALVLVSMLALGTLAEAQTETCTVAPRERQNCGFPGVTPSQCANKGCCFDDTVRGVPWCFYPNTIDVPPEEECEF

SEQ ID NO: 5 - RNA, 21 nt, Human
ugagaugaagcacuguagcuc

SEQ ID NO: 6 - RNA, 22 nt, Human
ugucaguuugucaaauacccca

SEQ ID NO: 7 - RNA, 22 nt, Human
uguaaacauccuugacuggaag

SEQ ID NO: 8 - RNA, 22 nt, Human
uucccuuugucauccuaugccu

SEQ ID NO: 9 - RNA, 22 nt, Human
uucaaguaauccaggauaggcu

SEQ ID NO: 10 - RNA, 22 nt, Human
uguaaacauccucgacuggaag

SEQ ID NO: 11 - RNA, 22 nt, Human
uguaaacauccuacacucagcu

SEQ ID NO: 12 - RNA, 21 nt, Human
uaaagugcugacagugcagau